(19) United States
(12) Patent Application Publication
(10) Pub. No.: US 2011/0044929 A1
(43) Pub. Date: Feb. 24, 2011

(54) FORMULATIONS AND METHODS FOR TREATMENT OF INFLAMMATORY DISEASES

(75) Inventors: John Joseph REDDINGTON, Burlingame, CA (US); Mary L. THIESSE, Burlingame, CA (US); Isabella PIESLAK, Atherton, CA (US)

Correspondence Address: Sughrue Mion/VICAL, 2100 Pennsylvania Avenue, N.W., Washington, DC, 20037, US

(73) Assignee: Vical Incorporated, San Diego, CA (US)

(21) Appl. No.: 12/862,692

(22) Filed: Aug. 24, 2010

Related U.S. Application Data
(62) Division of application No. 11/575,968, filed on Mar. 23, 2007, now abandoned.
(60) Provisional application No. 60/613,301, filed on Sep. 27, 2004.
(60) Provisional application No. 60/681,855, filed on May 17, 2005.

(51) Int. Cl. A61K 31/77; A61P 29/00

(52) U.S. Cl. 424/78.3

(57) ABSTRACT

The present inventors have developed a novel composition and method for inhibiting inflammation and treating of symptoms of tissue ischemia, including that associated with peripheral and cardiac vascular disease by local administration of a pharmaceutical composition including an effective amount of a poloxamer.

### RELATED APPLICATIONS

This is a submission under 35 §U.S.C. 371 of an application designating the U.S. and filed on Sep. 27, 2005 as PCT/US2005/034790, which claims priority to U.S. Provisional Patent Applications 60/613,301, filed Sep. 27, 2004, and 60/681,855, filed May 17, 2005; all three applications are hereby incorporated by reference as if fully set forth.

### TECHNICAL FIELD

The invention relates to formulations and methods for the treatment of inflammatory disease and tissue ischemia. The invention relates in particular to reducing inflammation and ischemia through the local administration of pharmaceutical compositions of non-ionic polymers.

### BACKGROUND OF THE INVENTION

Inflammation has recent emerged as a primary pathogenic mechanism that links cardiovascular risk factors and vessel dysfunction and injury associated with several vascular diseases. This is exemplified by atherosclerosis, a progressive disease characterized by the accumulation of lipids in large arteries. Elevated blood levels of inflammatory mediators such as interleukin (IL)-6, IL-8, IL-1β, monocyte chemoattractant protein 1 (MCP-1), tumor necrosis factor α (TNF-α), and surrogate markers of inflammation (e.g. soluble vascular adhesion molecule-1 (VCAM-1)) have been proposed as gauges of atherosclerotic risk. Further markers of atherosclerotic risk include high sensitivity C-reactive protein (hs-CRP) and serum amyloid A (SAA), which are products of hepatic stimulation by IL-6. Areas of the macro and microvasculature that are not associated with overt lesion development also assume the inflammatory phenotype characterized by oxidative stress and endothelial cell activation.

Major cellular participants in atherosclerosis include monocytes, macrophages, activated vascular endothelium, T lymphocytes, platelets and smooth muscle cells. Injury to vessel walls, including that induced by cigarette smoking, hypertension, atherogenic lipoproteins, and hyperglycemia, results in secretion of leukocyte soluble adhesion molecules that promote monocyte attachment to endothelial cells, as well as chemotactic factors that encourage migration of monocytes into the subintimal space. Transformation of these monocytes into macrophages that then take in cholesterol lipoproteins resulting in fatty streak initiation. Further attraction and accumulation of macrophages, mast cells, and activated T cells promote growth of an atherosclerotic lesion. Cardiovascular disease (CVD), including coronary artery disease (CAD) and peripheral vascular disease (PVD), is a sequela to atherosclerosis.

Peripheral vascular disease (PVD) refers to diseases of blood vessels outside the heart and brain, most commonly affecting the arteries that supply the lower extremities. Peripheral arterial disease (PAD) is an example of PVD and is a condition similar to coronary arterial disease (CAD) and carotid artery disease. In PAD (also known as peripheral arterial occlusive disease, “PAOD”), fatty deposits build up along artery walls and affect blood circulation, primarily in arteries leading to the legs and feet. Narrowing of the vessels that carry blood to leg and arm muscles is a typical cause of PAD with single or multiple stenosis and/or occlusion of the iliac-femoral-popliteal arterial axis determining a reduction of the perfusion of the muscles and the skin of the lower limbs and thus a progressive tissue ischemia.

Ischemia is a medical term describing a shortage of blood supply to an organ or tissue of the body. Ischemia typically results from narrowing or obstruction in the arteries that supply oxygen-rich blood to the tissues. Severe and prolonged ischemia leads to death of the affected tissue (infarction). Intermittent claudication, exhibited as lower extremity pain, cramping, numbness or fatigue during exercise relieved with rest, occurs in early stages of the disease. Approximately one-third to one-half of PAD patients suffer from intermittent claudication (IC), classically defined as pain in one or both legs that occurs with walking or exertion, does not resolve with continued activity, and abates upon rest or reduction in walking pace.

Coronary artery disease (CAD) refers to diseases of the blood vessels supplying oxygenated blood to the musculature of the heart (myocardium) resulting in cardiac ischemia. Narrowing or occlusion of one or more of the coronary arteries results in cardiac ischemia. Transient ischemia resulting from a failure of the blood supply to meet demands placed on the heart by increased physical activity or other stress-results in angina or chest pain. Severe or total obstruction of blood flow may result in death of heart muscle commonly referred to as myocardial infarction (heart attack). Heart disease is the leading cause of death in the United States. Cardiac ischemia is currently treated through the use of medication and physical conditioning to reduce the heart's oxygen demands or with drugs, angioplasty or bypass surgery to improve blood flow to the heart.

The current therapeutic options available to patients with symptomatic IC are primarily exercise, pentoxifylline, and cilostazol. Cilostazol (Pietal®) is a Type III phosphodiesterase inhibitor that increases intracellular cyclic adenosine monophosphate levels and promotes the release of prostaglandin I2. At the recommended dosage of 100 mg twice/day, cilostazol has been shown to improve peak walking time. However, this vasodilator drug does not result in biologic modification of the underlying disease, and the symptoms characteristically return on cessation of the drug. In addition, in clinical trials evaluating this agent, there is high incidence of side effects such as headaches, palpitations, and gastrointestinal disturbances.

Novel approaches to treating PAD include stimulating small vessel growth by delivery of angiogenic proteins or genes encoding angiogenic agents. The former approach, using delivery of recombinantly manufactured growth factors, has been shown to be effective in inducing an angiogenic response in a variety of animal models of acute limb and coronary ischemia, sometimes with the use of a single dose of an agent. (Takeshita S, et al. J Clin Invest (1994) 93:662-70; Harada K, et al. Am J Physiol (1996) 270:H1791-802; Lazarous D F, et al. Circulation (1996) 94:1074-82). Angiogenic proteins have been administered to humans in clinical trials, but these studies have yielded only modest evidence of efficacy. Potential systemic toxicities that limit the dose, coupled with the short half-life of the factors tested, may have limited effectiveness in these trials. (See Yancopoulos G D, et al. Nature (2000) 407: 242-248; Post M J and Simons M. Drug Discovery Today (2001) 6: 769-770).

Despite recent advances in therapeutic modalities for treatment of inflammatory disease including cardiovascular disease, there remains a further need for the identification of compositions and methods that are effective in reducing the severity of symptoms and improving the quality of life in affected patients without undesirable side effects. Furthermore, for the treatment of cardiovascular disease, drugs resulting in vasodilation or that stimulate angiogenesis may be considered a work around that may ameliorate symptoms of atherosclerosis but without affecting root pathogenic mechanisms such as inflammation. However, anti-inflammatory drugs such as corticosteroids have serious side effects. The COX-2 inhibitors, although selectively inhibiting inflammation, have been recently shown to have limiting side effects in many individuals. What are needed are compositions and methods for reducing inflammation while having a greater margin of safety.

### SUMMARY OF THE INVENTION

The present inventors have developed a novel approach for treatment of symptoms and inflammatory components of diseases, including those resulting in tissue ischemia, through the local extravascular administration of certain poloxamer formulations in affected areas. In one embodiment, the poloxamer is locally administered for deposition in an extravascular tissue by intramuscular, intravascular and/or intracapsular injection.

In one embodiment, the tissue ischemia is associated with peripheral vascular disease and the poloxamer is locally delivered by a plurality of intramuscular depot injections. In another embodiment, the polymer is locally administered in a depot injection for prolonged residence in and release from, an extravascular tissue after intramuscular injection.

In one embodiment of the invention, composition and methods are provided for control of inflammation mediated by IL-6 and/or IL-8 and/or MCP-1 in inflammatory sites by local administration of poloxamer-188 in such a way that the poloxamer is deposited for prolonged release from an extravascular tissue by intramuscular, intravascular and/or intracapsular injection. By depositing the polymer in an extravascular compartment, the half-life and effective presence of the polymer in the body is greatly extended such that a prolonged effect can be obtained.

In one embodiment of the invention, poloxamer-188 is administered by direct injection or pressure induced extravasation to the heart muscle thereby enabling a depot for prolonged release in the treatment of coronary artery disease. In one embodiment, a medicament including poloxamer 188 is manufactured for delivery by retrograde venous infusion through a balloon catheter placed in a vein draining into a coronary sinus with sufficient pressure to result in extravasation of the medicament into cardiac tissue. The vein draining into the coronary sinus is selected from the group consisting of a great cardiac vein (GCV), middle cardiac vein (MCV), posterior vein of the left ventricle (PVLV), anterior interventricular vein (AIV), and any of their side branches.

In one embodiment of the invention, poloxamer-188 is administered for the treatment of inflammation including atherosclerosis, bursitis, synovitis, tendonitis, perarticular disorders, rheumatoid arthritis, spondyloarthropathies, scleroderma (systemic sclerosis), Sjogren's Syndrome, polymyositis, dermatomyositis, systemic vasculitides, polymyalgia rheumatica, temporal arteritis, idiopathic multifocal fibrosclerosis, psoriasis, pericarditis, and systemic diseases in which arthritis is a feature.

In another embodiment of the invention, poloxamer-188 is administered for the treatment of injury induced inflammation including post-surgery, acute injury, and inflammation associated with surgical implants (joint, breast, etc.). In one embodiment the poloxamer is administered in conjunction with the implantation of a surgical prosthesis. Alternatively, the prosthesis is manufactured to comprises a quantity of the poloxamer, whereby the poloxamer is gradually released from the prosthesis.

In anther embodiment of the invention, poloxamer-188 is administered for the treatment of inflammation by local administration to the affected site in peritonitis, otitis externa, cystitis, chronic enterocolitis (a.k.a. Crohn's disease), mucositis (post-irradiation or chemo), pleuritis, vaginitis, conjunctivitis, and rhinitis/sinusitis.

In anther embodiment of the invention, poloxamer-188 is administered for the treatment of inflammation by local administration to the affected site in inflammatory skin conditions such as psoriasis, urticaria and angioedema, drug sensitivity rashes, pruritis, nodules and atrophic diseases, dermatitis including contact dermatitis, seborrheic dermatitis, chronic dermatitis, eczyma, photodermatoses, papulosquamous diseases, figurate erythemas, and macular, papular vesiculobullous and pustular diseases.

In one embodiment of the invention, poloxamer-188 is used in the treatment of gout by inhibition of production of IL-8 induced by sodium urate crystals.

In one embodiment, a poloxamer formulation. is disclosed that provides for treatment of symptoms of inflammation and ischemia in a peripheral limb, in cardiac muscle, in the kidney associated with renal vascular disease, ischemia associated with cerebral vascular disease, wound healing, non-union fractures associated with ischemia, avascular necrosis of the femoral head, diabetic neuropathy, erectile dysfunction, mesenteric ischemia, and celiac access ischemia. The formulation is administered by local delivery for example through intramuscular injection in the case of peripheral limb and cardiac muscle ischemia.

In one embodiment, the formulation is a pharmaceutical composition for treatment of inflammation by local administration to an affected tissue comprising an effective amount of a poloxamer-188 and a pharmaceutically acceptable carrier. Administration into an affected tissue includes administration into relatively normal tissues adjacent or leading to affected areas, including for example, administration to a thigh muscle where symptoms of inflammation and/or of ischemia are felt in the lower calf.

In one embodiment, the present invention provides a pharmaceutical composition for use in the treatment of inflammation in muscle, such as in a limb, that lessens one or more symptoms of peripheral vascular disease, including ischemia. In a further embodiment the composition is deposited in a plurality of individual doses in a novel, defined ring dosing pattern. For example, in the limb, the pattern of injections is such that a series of depositions of the formulation is in rings around the affected limb thus treating from proximal to distal and extending from a relatively non-ischemic region to areas of more pronounced ischemia (e.g. the injection pattern would begin in the muscle tissue that is well perfused with oxygenated blood (above the ischemic zone) and proceed well into the tissue with poor perfusion and an inadequate supply of oxygenated blood).

In one embodiment, a method of treatment of inflammation resulting in a symptom of peripheral vascular disease is provided that includes local intramuscular administration of a formulation comprising a poloxamer-188. Local intramuscular administration can be effected by injection into the muscle or by a vascular approach where the formulation is introduced into a local isolated portion of the vascular tree that perfuses the affected tissue and is extravasated from the vasculature by pressure. Once outside of the vasculature, the polymer is tissue resident for a prolonged period thus continuing to. exert a beneficial effect.

In one embodiment, the poloxamer is present in the formulation at a concentration of between 0.1 and 100%. In another embodiment the poloxamer is present at a concentration of less than 20% w/v in the formulation.

In one embodiment the non-ionic polymer is a poloxamer having a hydrophilic component of about 80% or greater and a hydrophobic molecular, weight between 950 and 4000 daltons, such as for example a poloxamer that has a flakeable solid physical form. In one embodiment the poloxamer is a poloxamer-188.

In one embodiment the poloxamer has the copolymer structure, physical form and surfactant characteristic of poloxamer-188 and is present in the formulation at a concentration of between about 0.1 and 20% w/v. In another embodiment the poloxamer-188 is present at a concentration of about 1-15%.

In one embodiment, the formulation includes an aqueous solution of poloxamer-188 at a concentration of about 50 mg/ml (5%) w/v and may further include one or more pharmacologic excipients.

In one embodiment of the invention, the poloxamer containing composition is lyophilized for storage and is rehydrated prior to administration.

In one embodiment, the polymer is packaged in a set of individual syringes, each syringe containing a volume to be administered through a single injection, such as through the skin and into a muscle tissue for multiple depot delivery of the polymer so that the polymer is tissue resident from each depot site for a prolonged period of time. In one embodiment, the volume per syringe or unit dose is determined on the basis of the anatomy of the administration site as well as the desired distribution area and the desired residence time for depot of poloxamer.

For purposes of this invention, “depot” is not limited to a visually observable mass of poloxamer but rather a quantity that is present in the tissue in a locally higher concentration for an extended period of time, i.e. a period of time exceeding that which. would be provided by intravascular administration. In one embodiment, each syringe in. the set is prepackaged to contain approximately 1-10 ml with each syringe in the set to be used for a single penetration through the skin. In another embodiment, each syringe is prepackaged to contain approximately 0.5-5 ml with each syringe in the set to be used for a single penetration through the skin. The poloxamer solution in each individual syringe can be delivered in either: a single depot; intermittent deposition at multiple sites along the needle track; or essentially constant steady deposition as the needle is withdrawn. In one embodiment, a depot administration into tissue of poloxamer 188 is provided in which a total dose of from 0.24-13 grams of poloxamer is delivered.

In one embodiment, syringes comprising an aqueous solution of a poloxamer are provided, wherein said syringe is suitable for depot delivery of said poloxamer to treat tissue ischemia and/or inflammation. In one embodiment, poloxamer is doposited at a. concentration of between 0.1 and 100% w/v. In another embodiment, each syringe comprises approximately 1 to 4 ml of an aqueous solution of between 0.1 and 25% w/v. In one embodiment, the poloxamer has a hydrophilic content of about 80% or greater and a hydrophobic molecular weight between 950 and 4000 daltons. In one preferred embodiment, the poloxamer has a copolymer structure, physical form and surfactant characteristic of a poloxamer 188 and is present at a concentration of between about 0.1 and 20% w/v, preferably between about 1 and 6% w/v.

In one embodiment, the syringes are prepackaged with approximately 2 ml per syringe and in a full set for the use of one prefilled syringe for each of multiple depot injections. In one embodiment the syringes are prepackaged with approximately 1 ml per syringe and in a full set for the use of one prefilled syringe for each of multiple depot injections. In one embodiment involving a plurality injections into the muscle delivered at a single treatment, each syringe is suitable for intramuscular depot delivery of the poloxamer to treat peripheral vascular or cardiovascular disease and a syringe set is provided that includes from approximately 12 to 42 individual prefilled syringes to be used to treat one patient in a single treatment.

In one embodiment, the polymer is an approximately 5% poloxamer solution. In one preferred embodiment, the poloxamer is a poloxamer-188 provided in the following formulation: a sterile solution of 5% w/v poloxamer-188, 5 mM Tris-HCl pH 8.0, and 0.9% w/v sodium chloride injection, USP. In one embodiment, a 2-5 ml Type 1 borosilicate glass syringe is prefilled with the sterile poloxamer formulation and delivered using a 25 gauge, 3 inch spinal syringe.

In one embodiment, a kit is provided that includes a set of 12 to 42 individual syringes with instructions for administration. In an alternative embodiment, a kit is provided that includes bottle of lyophilized poloxamer in sufficient quantity for multiple dose administration together with suitable diluent for reconstituting the poloxamer. The kit may or may not include a set of unfilled syringes adapted to the site of administration.

In one embodiment, bulk sterile solutions are produced containing, for each liter of formulation, 50 grains of poloxamer-188, 0.28 grains of Tris Base USP, 0.44 grams of Tris-HCl, and 9 grams of NaCl USP, dissolved in water.

In one embodiment of the invention pharmaceutical formulations and methods are provided for inhibiting inflammation mediated at least in part by at least one of IL-6, IL-8, MCP-1. In one embodiment the inflammation is associated with symptoms of intermittent claudication and the poloxamer is administered by multiple intramuscular injections of an aqueous solution of poloxamer-188 into the affected limb. In a further embodiment, the multiple injections are made in successive injection rings in a flow to no-flow pattern.

In a further embodiment the anti-inflammatory effects of extravascular polymer deposition are combined with one or more further agents that are able to stimulate the growth and maturation of new collateral vessels in an ischemic tissue.

The invention is further taught and exemplified by the following details.

### BRIEF DESCRIPTION OF THE DRAWINGS

A better understanding of this invention can be obtained when the following detailed description of the preferred embodiments is considered in conjunction with the following drawings.

FIG. 1A. Depiction of ELISA results for IL-6 production by normoxic HUVEC cells with various treatments.

FIG. 1B. Depiction of ELISA results for IL-6 production by hypoxic HUVEC cells with various treatments.

FIG. 2A. Depiction of ELISA results for IL-8 production by normoxic HUVEC cells with various treatments.

FIG. 2B. Depiction of ELISA results for IL-8 production by hypoxic HUVEC cells with various treatments.

FIG. 3A. Depiction of protein macroarray results for MCP-1 production by hypoxic HUVEC cells with various treatments.

FIG. 3B. Depiction of protein macroarray results for MCP-1 production by hypoxic HSMM cells with various treatments.

FIG. 4A. Depiction of ELISA results for adenosine production by normoxic HUVEC cells with various treatments.

FIG. 5. Grid representing poloxamer and reverse poloxamer characteristics.

FIG. 6. Chemical structures of poloxamers and reverse poloxamers.

FIG. 7. Characteristics of useful poloxamers for muscle delivery.

FIG. 8. Anatomy of the lower limb.

FIG. 9. Depiction of administration by needle injection into the muscle.

FIG. 10. Depiction of ring pattern of administration by needle injection into the muscle.

FIG. 11. Exercise tolerance results from Phase I safety trial.

### DETAILED DESCRIPTION OF THE PREFERRED EMBODIMENTS

The rationale that lead to the present invention began with efforts to develop a pharmaceutical formulation for delivery of the Del-1 gene for the in situ production of the angiogenic Del-1 protein in patients suffering from tissue ischemia. In the course of these efforts, the present inventors surprisingly found that certain poloxamers have differential effects on specific proinflammatory cytokines and chemokines.

Poloxamer-188 treatment was found to result in differential release of several inflammatory mediators from endothelial cells: IL-6, IL-8 and monocyte chemotractant protein-1 (MCP-1). Specifically, it was found that poloxamer-188 has the property of inhibiting the release of IL-6 and IL-8 from endothelial cells. Poloxamer-188 was also found to inhibit the release of MCP-1 from skeletal muscle myocyte cells. When treated with compounds other than poloxamers, human vascular endothelial (HUVEC) cells in culture increasingly release IL-6 and IL-8 into the medium over time under both normoxic and hypoxic conditions. Poloxamer-235 dramatically increased IL-6 and IL-8 production from HUVEC cells compared to controls: In contrast, poloxamer-188 was found to selectively inhibit the production of IL-6 and IL-8 by HUVEC cells under either normoxic or hypoxic conditions.

IL-6 and IL-8 are among the proinflammatory cytokines (interleukin-1 [IL-1], IL-6, IL-8, IL-12, IL-15, IL-18, and tumor necrosis factor-α [TNF]) that are typically in functional equilibrium with the anti-inflammatory cytokines (including IL-4, IL-10, IL-11, IL-13) and endogenous cytokine inhibitors (IL-1 receptor antagonist [IL-1ra], IL-18 binding protein, and soluble receptors for IL-1 and TNF). Disequilibrium of this balance results in inflammatory mediated disease.

Interleukin 6 (IL-6), originally identified as a B-cell differentiation factor, is now known to be an important regulator, not only in immune responses and inflammation, but also in hematopoiesis, liver and neuronal regeneration. IL-6 stimulates B-lymphocyte proliferation and neutrophil production and is produced by many cells including T-lymphocytes, macrophages, monocytes, endothelial cells, and fibroblasts. Increased IL-6 levels are associated with several diseases, including rheumatoid arthritis (RA), systemic-onset juvenile chronic arthritis (JCA), osteoporosis, psoriasis, inflammatory bowel disease, multiple sclerosis and various types of cancer. (Heinrich P C, et al. Biochem J. 374 (Pt 1) (2003)1-20).

IL-8 is chemotactic for all known types of migratory immune cells. IL-8 differs is unique in its role as a specific activator of neutrophil granulocytes. IL-8 is produced by macrophages, fibroblasts, endothelial cells, keratinocytes, melanocytes, hepatocytes, chondrocytes, and a number of tumor cell lines. IL-8, together with IL-1 and IL-6, are thought to participate in the pathogenesis of chronic polyarthritis as excessive amounts of IL-8 are found in synovial fluids. Neutrophil activation by IL-8 may enhance migration of cells into the capillaries of the joints where the cells can leave the capillaries and enter the surrounding tissues. Reduced production of IL-8 is expected to decrease migration of neutrophils and monocytes (via IL-8 chemotaxis) to the vessel wall thus dampening the chronic inflammatory process that is an underlying cause of atherosclerosis disease progression. IL-8 is induced by sodium urate crystals and thus in one embodiment of the invention, poloxamer-188 is used in the treatment of gout.

Monocyte chemoattractant protein-1 (MCP-1) is a chemotactic chemokine that displays immunoregulatory functions and may be involved in Th1 subset differentiation by modulating the differentiation of monocytes into DCs. Although initially identified as a monocyte-specific chemoattractant, MCP-1 has now been shown to attract activated. T cells, NK cells, and basophils, as well as monocytes. MCP-1 is postulated to be involved in the pathogenesis of diseases characterized by mononuclear cell infiltration including rheumatoid arthritis and bronchial asthma. (Omata N, et al. J Immunol. 169(9) (2002) 4861-6). MCP-1 is also highly expressed by postinjured muscle and has been postulated to play a role in traumatic muscle injury/recovery. (Summan M, et al. J Interferon Cytokine Res. 23(5)(2003) 237-45).

In one embodiment of the invention, compositions and methods are provided for control of inflammation mediated by IL-6 and/or IL-8 and/or MCP-1 in inflammatory sites by local administration of poloxamer-188 in such a way that the poloxamer is deposited for prolonged release from an extravascular tissue by intramuscular, intravascular and/or intracapsular injection. By depositing the polymer in an extravascular compartment, the half-life and effective presence of the polymer in the body is greatly extended such that a prolonged effect can be obtained.

In atherosclerosis, elevated blood levels of inflammatory mediators such as interleukin (IL)-6, IL-8, IL-1β, monocyte chemoattractant protein 1 (MCP-1), tumor necrosis factor α (TNF-α), and surrogate markets of inflammation (e.g. soluble vascular adhesion molecule-1 (VCAM-1)) have been proposed as gauges of atherosclerotic risk. Remarkably, poloxamer-188 selectively affects several of these critical pro-inflammatory cytokines. Reduced production of IL-6 by the expansive endothelial component of the peripheral vasculature is expected to decrease the release of IL-6 induced CRP in the liver.

The IL-8 like cytokine GRO (growth regulated cytokine) also appears to be differentially regulated by poloxamer-188 treatment and studies are on-going on this effect. GRO, also known as melanoma growth stimulatory activity (MGSA), describes a family of closely related chemokines including GRO-alpha (also known as neutrophil activating peptide-3), GRO-beta and GRO-gamma. The three GRO genes are expressed in a tissue-specific manner. Although predominantly found in monocytes after cell activation, they are also expressed in fibroblasts, endothelial cells, synovial cells, and several tumor cell lines. GRO has inflammatory and growth-regulating properties and is a potent chemoattractant for neutrophils. GRO proteins are functionally related to IL-8 and also bind to the same receptor.

In one embodiment of the invention, poloxamer-188 is administered for the treatment of inflammation including atherosclerosis, bursitis, tendonitis, synovitis, perarticular disorders, rheumatoid arthritis, spondyloarthropathies, scleroderma (systemic sclerosis), Sjogren's Syndrome, polymyositis, dermatomyositis, systemic vasculitides, polymyalgia rheumatica, temporal arteritis, idiopathic multifocal fibrosclerosis, psoriasis, pericarditis and systemic diseases in which arthritis is a feature.

Systemic diseases that may ultimately include an arthritis component include autoimmune hepatitis, primary biliary cirrhosis, Whipple's disease, pancreatic-arthritis syndrome, hemophilia, hemoglobinopathies, hypogammaglobulinemia, celiac disease, hemochromatosis, diabetes mellitus, thyroid disorders, parathyroid disorders, acromegaly, hyperlipoproteinemia, Paget's disease, and hypertrophic osteoarthropathy.

In another embodiment of the invention, poloxamer-188 is administered for the treatment of injury induced inflammation including post-surgery, acute injury, and inflammation associated with surgery including that involved with surgical implants (joint, breast, etc.). In one embodiment, poloxamer 188 constitutes or is included in the fluid that fills breast prostheses (implants) such that any poloxamer that leaks or gradually escapes from the implant will suppress inflammatory reactions that result in scarring, influx of inflammatory cells, capsule formation and hardening of the implant. Animal studies disclosed herein indicate that poloxamer 188 is able to inhibit both inflammatory and foreign body reactions.

In another embodiment of the invention, poloxamer-188 is administered for the treatment of inflammation by local administration to the affected site in peritonitis, otitis externa, cystitis, chronic enterocolitis (a.k.a. Crohn's disease), mucositis (post-irradiation or chemo), pleuritis, vaginitis, conjunctivitis, and rhinitis/sinusitis.

In another embodiment of the invention, poloxamer-188 is administered for the treatment of inflammation by local administration to the affected site in inflammatory skin conditions such as psoriasis, urticaria and angioedema, drug sensitivity rashes, pruritis, nodules and atrophic diseases, dermatitis including contact dermatitis, seborrheic dermatitis, chronic dermatitis, eczyma, photodermatoses, papulosquamous diseases, figurate erythemas, and macular, papular vesiculobullous and pustular diseases.

In one embodiment of the invention, poloxamer-188 is used in the treatment of gout by inhibition of production of IL-8 induced by sodium urate crystals.

In one embodiment, a poloxamer formulation is disclosed that provides for treatment of symptoms of inflammation and ischemia in a peripheral limb, in cardiac muscle, in the kidney associated with renal vascular disease, ischemia associated with cerebral vascular disease, wound healing, non-union fractures associated with ischemia, avascular necrosis of the femoral head, diabetic neuropathy, erectile dysfunction, mesenteric ischemia, and celiac access ischemia. The formulation is administered by local delivery for example through intramuscular injection in the case of peripheral limb and cardiac muscle ischemia.

Underlying Studies: The development of Del-1 for therapeutic angiogenesis was based on results from in-house preclinical studies using angiogenic growth factors employing both protein and gene based strategies. Del-1 (Developmentally regulated Endothelial Locus-1) is an endothelial cell stimulating protein expressed during embryological development of the vascular tree. (Hidai C, et al. Genes Dev (1998 Jan. 1)12(1):21-33). Postnatally, Del-1 is also expressed at sites of angiogenesis. Del-1 supports the adherence and migration of endothelial and vascular smooth muscle cells, mediated via binding to the αvβ3 integrin receptor.

Repeated intramuscular injections of Del-1 protein demonstrated increased vascular perfusion in a murine hind limb ischemia model. A gene-based approach to Del-1 delivery using a plasmid vector was developed for the purpose of enhancing relatively sustained local concentrations with a consequent reduction in systemic exposure to the angiogenic growth factor while at the same time avoiding known adverse effects that may arise with the use of a viral platform.

Results from preclinical studies with recombinant murine Del-1 protein and with formulated Del-1 plasmid compared favorably to results obtained with bFGF and VEGF₁₆₅. In-house research provided for selection of the non-ionic polymer poloxamer 188 as an important constituent for a pharmaceutical formulation of the Del-1 gene encoded on plasmid DNA. The poloxamer formulation was developed after considerable research to provide for a compound that would give increased expression over DNA in saline. In a mouse model of hind limb ischemia, injection of formulated Del-1 plasmid was shown to increase capillary density and to increase treadmill run time compared with a formulated empty vector. In a rabbit model of hind limb ischemia, injection of Del-1 plasmid was found to increase collateral vessel formation and CD-31 expression compared with a formulated empty vector. No toxicity was directly attributable formulated human (h) Del-1 plasmids in preclinical animal studies. The results of preclinical animal studies did not suggest a significant effect on collateral vessel formation or increased exercise tolerance attributable to the poloxamer, although the poloxamer was significantly better than saline in increasing expression of the plasmid DNA.

On the basis of safety and efficacy in preclinical animal studies, a human Phase I dose escalation trial designed to determine the maximum tolerated dose was conducted in which twenty-seven human subjects with PAD received up to 28 IM injections of poloxamer-188 formulated Del-1 administered to one leg in one procedure. The study formulation, VLTS-589, consisted of 1 mg/ml Del-1 encoding plasmid DNA, 50 mg/ml w/v poloxamer 188 (Spectrum Chemical, Poloxamer 188, NF), 0.28 mg/ml w/v Tris, and 0.44 mg/ml Tris-HCl in an aqueous saline solution. Twenty-six subjects completed the study according to the protocol. The dose delivered to subjects ranged from 3 mg (single injection) to a maximum of 84 mg (28 injections) of VLTS-589. Ten subjects received the top dose of 84 mg of VLTS-589. No serious adverse events related to the study drug were observed among the subjects who received VLTS-589. On the basis of positive safety results and a trend supporting increased efficacy with increasing dose as depicted in FIG. 11, a Phase II trial was initiated.

The Phase Ha double-blind, placebo-controlled trial was designed to determine the safety and efficacy of VLTS-589 compared with “placebo” in 105 subjects with PAD. The “placebo” represented an identical polymer formulation to VLTS-589 but lacked the plasmid DNA. Thus the “placebo” was essentially an aqueous pharmaceutically acceptable solution of 5% poloxamer-188. The subjects were randomized to receive a single treatment of VLTS-589 or placebo administered as 21×2 mL IM injections bilaterally into the lower extremities during one procedure. The dose of VLTS-589 was 84 mg (42 mg in each leg).

Upon opening of the code at the conclusion of the double blind trial period, the present inventors surprisingly discovered and appreciated that a non-ionic polymer, in this case poloxamer 188, was able to relieve certain of the symptoms of PAD including the pain of intermittent claudication in a significant number of patients. The ability to ameliorate one or more symptoms of PVD using a non-ionic polymer represents a significant advance in the medical treatment of this disease. In particular, a significant number of patients were able to increase their peak walking time and their ankle brachial index (ABI). The increase in walking time, as well as the increased tissue perfusion manifest by the improved ABI, may further stimulate the development of new vessels, thus amplifying the effect initiated by the polymer treatment and providing further relief of the ischemic manifestations of the disease.

Investigations into the mechanism of the poloxamer effect were undertaken in light of the inventor's unifying synthesis of information relating to inflammation in cardiovascular and other diseases. It has now been remarkably discovered that poloxamer-188 selectively inhibits elaboration of certain inflammatory mediators and that this property differs considerably from that of another poloxamer, poloxamer-235 (Pluronic P85). Thus, the present invention provides a novel modality for the treatment of a variety of diseases having an inflammatory component.

In hypercholesterolemic animals, elevated systemic markers of inflammation, impaired dilatory capacity of arterioles, and increased blood cell recruitment in post-capillary venules appear to be linked (either directly or indirectly) to endothelial cell activation and are observed long before lesion development in large arteries. (Singh U and Jialal. I. Ann. N.Y. Acad. Sci. 1031 (2004) 195-203; Stokes K Y and Granger D N. J. Physiol. 562.3 (2004) 647-553). The manifestations of endothelial cell dysfunction appear to be linked to oxidative stress and imbalance between superoxide and nitric oxide (NO) in vascular endothelial cells. The endothelial oxidative stress is largely due to activation of superoxide-producing NAD(P)H oxidase in arteries.

Increased VCAM expression by the endothelial cell mediates a critical step in atherosclerotic lesion formation, namely the recruitment of leukocytes to the vessel wall. This not only leads to circulating leukocyte stimulation but also platelet activation. The activated platelets further favor the recruitment of leukocytes onto endothelial cells overlaying plaques by forming platelet-monocyte aggregates and by depositing chemokines. Monocytes promote the peroxidation of lipids, such as low-density lipoproteins (LDLs) through the generation of reactive oxygen species. Chemotaxis and entry of the monocytes into the subendotheial space is promoted by monocyte chemoattractant protein-1 (MCP-1), IL-8, and a newly reported chemokine, fractaline: IL-6, a messenger cytokine, is secreted by the monocytes and endothelial cells where it activates receptors in the liver, leading to production of C-reactive protein (CRP). CRP is transported free in the plasma where it accumulates at the site of inflammation presumably by binding to oxidized phospholipids. Proposed atherogenic mechanisms involving CRP are largely based on cultured endothelial cell models. The proposed mechanisms include impaired production of nitric oxide (NO) and prostacyclin, and increased production of endothelin-1, various cell adhesion molecules, MCP-1, and IL-8. CRP has also demonstrated to promote monocyte adhesion and chemotaxis. Many of the inflammatory factors and cells induce vascular smooth muscle cell (VSMC) to migrate and subsequently proliferate to form the fibrous cap of the lesion.

Studies on the responses of the microvasculature to elevated blood cholesterol levels have revealed changes that are consistent with endothelial cell activation in both arterioles and postcapillary venules of several vascular beds. (Gauthier T W, et al. Atheroscler. Thromb. Vasc. Biol. 15 (1995) 1652-1659). These changes long predate the appearance of atherosclerotic plaques in large arteries. While vascular dysfunction is manifested differently between arterioles and venules, oxidative stress appears to be experienced by endothelial cells throughout the vasculature. Reactive oxygen species (ROS) signaling mechanism and superoxide-mediated inactivation of NO are frequently implicated in the altered endothelial cell-dependent processes in the microcirculation that accompany hypercholesterolemia. (Harrison D G and Ohara Y. Am. J. Cardiol. 75 (1995) 75-81B). NO stimulates cGMP generation in, and therefore relaxation of, adjacent smooth muscle cells. A likely result of the defective endothelium NO-dependent vasodilatory responses in hypercholesterolemia is impairment of blood flow regulation in different tissues. Venules appear to respond to hypercholesterolemia by decreasing the diameter of the adjacent arterioles via an NO-dependent mechanism that ultimately leads to reduced capillary flow. The reduction in capillary and overall tissue perfusion also appears to be neutrophil dependent. (Nellore K and Harris N R. Microcirculation 9 (2002) 477-485).

It has recently been speculated that the microcirculation may be an important source of the inflammatory signals that drive large vessel disease and it may contribute to the production of the circulating surrogate markers of inflammation that are detected in atherosclerotic patients. (Rattazzi M, et al. J. Hypertension 21 (2003) 1787-1803). Evidence for activation of endothelial cells, leukocytes and platelets in venules of several vascular beds, coupled to the involvement of immune cell-derived cytokines in the modulation of the microvascular responses to hypercholesterolernia, support this possibility.

If endothelial cell activation is a rate-determining factor in producing the systemic inflammatory response to hypercholesterolemia, and if this inflammatory phenotype is assumed by endothelial cells throughout the vasculature, then any consideration of the relative contributions of endothelial cells in large arteries and the microvasculature to this response should take into account the endothelial surface area of each vascular compartment.

In a 70 kg man, the estimated endothelial surface area that is associated with the atherosclerosis-prone aorta is 156 cm², while the larger vessels collectively are 3,333 cm². In contrast, a published surface area estimate is 361,337 cm² for the arterioles and 879,989 cm² for the venules. (Wolinsky, H. Circulation Research 47 (1980) 301-311). Thus, the microvasculature provides an area that is estimated to be at least 300 times larger in surface area than the larger vessels. Taken together, this information points to the microcirculation, where chronic endothelial cell injury occurs during hypercholesterolemia, as an integral contributor to the chronic inflammatory process that helps drive the progression of atherosclerosis.

Because the present inventors considered that any potential beneficial effect of poloxamer 188 on the observed improvement on peak walk time might be mediated through the endothelial cells of the vasculature and the skeletal muscle cells themselves, the potential direct effects of poloxamer-188 and other chemicals on these cell types was considered. Reviewing the scientific literature revealed a lack of information regarding the injection of poloxamers into solid tissue. Poloxamer-235 (BASF Pluronic P85), a poloxamer that has apparently been reported as an injectable delivery vehicle of chemotherapeutics into multiple drug resistant tumors, was report to cause the release of adenosine and ATP from cells. (Kabanov A V et al. J Control Release 91(1-2) (2003) 75-83; Batrakova E V, et al. Br J Cancer 85(12) (2001) 1987-1997). Poloxamer-235 has the following correlative nomenclature and structural characteristics: BASF Pluronic name: P85; BASF average molecular weight: 4600 D; Average number of POP units: 39.7; Average number of POE units: 52.3; weight % POE: ˜50%; molecular weight of POP: 2400; molecular formula: HO—(C₂H₄O)₂₇—(C₃H₆O)₃₉—(C₂H₄O)₂₇—H.

Adenosine and ATP have been shown to be vasodilatory, and recently, adenosine has been shown to be angiogenic. (Biaggioni I. Clin Pharmacol Ther 75 (2004) 137-39; Hein T W, et al. J Pharmacol Exp Ther 291 (1999) 655-64; Montesinos M C, et al. Am J Path 164 (2004) 1887-92; Adair T H. Hypertension 44 (2004) 1-30). Both adenosine and ATP have their affect on vascular endothelial cells to cause the observed biological events. The endothelial cell has repeatedly been implicated as playing a key role in the progression of atherosclerosis.

For this reason, studies were undertaken to determine the effect of certain poloxamers in vitro in human vascular endothelial cells (HUVEC) and human skeletal muscle myoblast (HSMM) cells under normoxic and hypoxic (5% O₂) conditions. Production of adenosine, cytokines, growth factors, or a combination of these biologically relevant molecules was measured after exposure to either poloxamer-188 (˜BASF Pluronic F-68), poloxamer-235 (˜BASF Pluronic P85), cilostazol, Del-1 protein, or medium alone. Monitoring for generation of adenosine was conducted by HPLC. When the effects of poloxamer-188, poloxamer-235, cilostazol, and Del-1 protein on HUVECs were compared under normoxic and hypoxic conditions with those of medium alone, it appeared that poloxamer-235 did lead to the higher levels of adenosine in the supernatant versus the other treatments. This increased level of adenosine was seen over time in both hypoxic and normoxic cells exposed to poloxamer-235. Cilostazol and Del-1 protein appeared the least stimulatory in adenosine production in these studies, while poloxamer-188 trended toward an intermediate level of release into the medium. Thus, the conclusion from the initial study was that poloxamer-188 does not appear to be as efficient as poloxamer-235 in causing the cells to release adenosine, and although it may contribute to a potential beneficial effect, it is probably not the only mechanism through which poloxamer-188 may be working.

Certain of the poloxamers have been reported to have effects that may be considered immunological. For example, poloxamer-188 has been reported to inhibit neutrophil migration chemotaxis and adhesion including to inflammatory loci. (Lane T A, Lamkin G E. Blood. 1986 Aug;68(2):351-4). A different poloxamer, CRL-1072 has been reported to enhance antimycobacterial activity of human macrophages though IL-8. CRL-1072 is a highly hydrophobic poloxamer having a mean molecular mass of polyoxypropylene (POP) chains of 3,500 Da each and POE chains of 200 Da each and is thus ˜10% polyoxyethylene (POE). CRL1072 appears to have been designed to be a molecularly pure analogue of poloxamer-331 (˜BASF Pluronic L101). It was found that human macrophages treated with CRL-1072 synthesized interleukin-8 (IL-8), tumor necrosis factor-alpha (TNF-alpha), and granulocyte-macrophage colony-stimulating factor (GM-CSF) in a dose-dependent manner. (Jagannath C, Pai S, Actor J K, and Hunter R L. J Interferon Cytokine Res. 1999 January; 19(1):67-76).

Interestingly, intraperitoneal injection of poloxamer-407 (a.k.a. PLURONIC F127) induces atheroscleorosis and forms the basis of one animal model for this disease. However, it has been recently reported that this is due to lipid derangements and not due to direct effects on endothelial cells and macrophages. Studies demonstrated that incubation of poloxamer-407 with human umbilical vein endothelial cells in culture did not influence either cell proliferation or interleukin-6 and interleukin-8 production over a concentration range of 0-40 microM. (Johnston T P, et al. Mediators Inflamm. 2003 June; 12(3):147-55).

Based on a perceived potential for an inflammatory component to the observed effect of intramuscular poloxamer in relieving symptoms of peripheral ischemia, monitoring for generation of over 40 cytokines and growth factors in various cell types was conducted using protein macroarrays and the results were confirmed using liquid phase ELISA. Surprisingly, it was found that poloxamer-188 differs significantly from poloxamer-235 in its effects on endothelial and skeletal muscle cells.

Protein Macroarrays and ELISAs: Human umbilical vein endothelial cells (HUVEC-Human umbilical vein endothelial cells, Cambrex, Cat #CC2617) are grown in EBM-2 (Endothelial cell basal medium-2, Cambrex, Cat #CC-3156), and EGM complete media-2 (EGM-2, Cambrex, Cat #CC-4176). Human skeletal muscle myoblasts cells (HSMM-Human skeletal muscle myoblasts cells, Cambrex, Cat #CC-2580T25) are grown in SkBM-2 (Skeletal muscle myoblast basal medium-2, Cambrex, Cat #CC-3246) and SkGM complete media (SkGM-2 BulletKit, Cambrex, Cat #CC-3245) in the T75 flasks to confluency of 70 to 90%.

HUVEC and HSMM cells are harvested after fourth population doubling from the time of purchase by trypsinization. The cells are suspended in appropriate complete medium and plated in a 60×15 culture dishes at the density of 10⁻⁶ cells per well and incubated for 24 hrs. The cells are fed with EBM (HUVEC cells) and SkBM (HSMM cells) culture medium containing 0.5% FCS for 24 hrs to growth-arrest the cells. After 24 hrs the cells are treated with 10 μM/L EHNA (Erythro-9-(2-Hydroxy-3-nonyl)adenine, Sigma, Cat #0114, to prevent degradation of adenosine to inosine), 10 μM/L dipyridamole (Sigma, Cat #D9766, to inhibit cellular adenosine uptake), 1 μM/L iodotubercidin (A.G Scientific, Inc. Cat #11005, to prevent incorporation of adenosine into AMP). Test solutions are designed to provide final concentrations in culture media of: 5% w/v poloxamer-188; 5% w/v poloxamer-235; 100 nM (5.2 ng/ml final) human del-1 protein; 20 pg/ml adenosine (Sigma, Cat #4036); and 10 μM (3.69 μg/ml final) cilostazol (Sigma, Cat #C-0737, stock dissolved in DMSO). Test solutions were added to culture dishes with some dishes remaining with just media as controls. One set of plates are incubated under hypoxic conditions such as 5% O₂, 5% CO₂, and 90% N₂ in a sealed chamber. The normoxic conditions are essentially normal air with added 5% CO₂. Cells are cultured for approximately for 2, 6, 12, 24 and 48 hrs and cells and supernatants are collected separately at each time point and stored at ⁻80° C. for the analysis.

After the supernatants are collected, the cells are washed 1× with PBS and the cell lysed by addition of 1 ml Lysis Buffer (Promega Lysis Buffer, Cat #E1941, plus Protease Inhibitor Cocktail, Calbiochem Cat #539134). Cells were scraped into the lysis buffer, disrupted by pipetting and transferred into microfuge tubes for freezing at ⁻80° C. After thawing and centrifuging at 10,000 RPM in a microcentrifuge for 2 minutes, the supernate was transferred to cryovials for storage at ⁻20° C.

Adenosine analysis was conducted by liquid chromatography using a Shimadzu VP System and a 2×20 mm Higgins Analytical Phalanx C₁₈ guard cartridge for assaying an injection volume of 25 μl. The mobile phase was 0.1% trifluoroacetic acid in water (A) and in methanol (B) and the gradient was 0-75% (B) in 2 minutes after a 0.5 minute wash and a flow rate of 400 μl/min. An Applied Biosystems/MDS SCIEX API 3000 Mass Spectrometer was used together with a TurboIonSpray interface at 400° C. in a positive ion ionization mode. The Q1/Q3 ions were 268.1/136.2 with 256.2/167.2 for Diphenhydramine and 272.1/215.2 for Dextromethorphan.

Adenosine receptors A2A and A2b were assessed by western blot using a Novex vertical gel apparatus and Novex pre-cast 10% Tris-Glycine gels (Novex #EC6075) according to standard techniques. Rabbit Anti-Canine Ata receptor Ab, (A2aR) affinity purified or Rabbit Anti-human A2bR IgG Affinity purified (Primary antibodies Alpha Diagnostics International) were used together with Goat Anti-Rabbit IgG (H+L)-HRP (secondary antibody Alpha Diagnostics International). ECL Reagents were obtained from Amersham (RPN2106).

Protein MacroArrays were conducted using commercial kits including RAYBIO Human Cytokine Antibody Array III (Cat No. H0108009) for supernate analysis and Human Cytokine Antibody Array 3.1 for cell lysate analysis (Cat. No. H0109809). Both arrays test for ENA-78, GCSF, GM-CSF, GRO, GRO-alpha, I-309, IL-1 alpha, IL-1beta, IL-2, IL-3, IL-4, IL-5, IL-6, IL-7, IL-8, IL-10, IL-12p40p70, IL-13, IL-15, IFN-gamma, MCP-1, MCP-2, MCP-3, MCSF, MDC, MIG, MIP-1 delta, RANTES, SCF, SDF-1, TARC, TGF-beta1, TNF-alpha, TNF-beta, EGF, IGF-1, angiogenin, oncostatin M, thrombopoietin, VEGF, PDGF BB, and leptin. Detection was via Biotin-Conjugated Anti-Cytokines and HRP-Conjugated Streptavidin. If serum containing conditioned media was required, serum was used as a control.

ELISA kits were used to detect IL-8 (R&D Systems, Cat #D8000C), human VEGF (R&D Systems, Cat #DVE00), human IL-6 (R&D Systems, Cat #D6050), and human MCP-1 (BioSource International, Cat #KHC1011). The assays were performed on conditioned supernatants and cell lysates collected at 2, 6, 12 and 24 hrs from HUVEC and from HSMM cells at 12, 24 and 48 hrs.

Samples of cell culture supernatants from HUVEC or HSMM cell lines incubated under normoxic or hypoxic (5% O₂) conditions and exposed to either poloxamer-188, Del-1 protein (Del-1), Cilostazol (CST), poloxamer-235, or adenosine were compared. As controls, cells were maintained with either normal medium, or medium containing 0.5% fetal bovine serum under normoxic or hypoxic conditions.

Selective Inhibition of IL-8 and IL-6 by Poloxamer-188: Using solid phase protein macroarrays (RayBiotech, Inc., Norcross, Ga.), of the 42 cytokines screened for in this assay system, qualitative results from culture supernatant yielded a visual and numeric difference in four cytokines, MCP-1, IL-6, IL-8, and the IL-8 like cytokine GRO (growth regulated cytokine). In HUVEC cells using the protein macroarrays analysis, poloxamer 188 appeared to suppress the release of IL-6, and IL-8 from HUVECs during the 24 hours of culture under normoxic and hypoxic conditions when compared to poloxamer-235. According to densitometry scans, IL-6 was dramatically stimulated by poloxamer-235 while of all the treatment groups including poloxamer-188, Del-1, CST and Adenosine, only poloxamer-188 reduced expression of IL-6 under both normoxic and hypoxic conditions. Similarly, IL-8 was dramatically increased by poloxamer-235 treatment. When MCP-1 release from HUVECs was analyzed, both poloxamer-188 and poloxamer-235 yielded similar results under both normoxic and hypoxic conditions. Negative control (Ctrl 0.5), Del-1 protein, cilostazol (CST) and adenosine treatments resulted in similar patterns of release for IL-6, IL-8 and MCP-1 into the medium from HUVECs under both normoxic and hypoxic conditions.

In HSMM cells using the protein macroarrays analysis, IL-6 release for all treatments was below the confidence threshold (˜8,000 units) for the assay system. IL-8 levels in the supernatants from all treatment groups under normoxic and hypoxic conditions were similar. However, MCP-1 levels in HSMM culture supernatants were lower in the poloxamer-188 groups than the other treatments under both normoxic and hypoxic conditions. As a consequence of the macroarray results, further emphasis was directed to MCP-1, IL-6, and IL-8, in particular using capture ELISA.

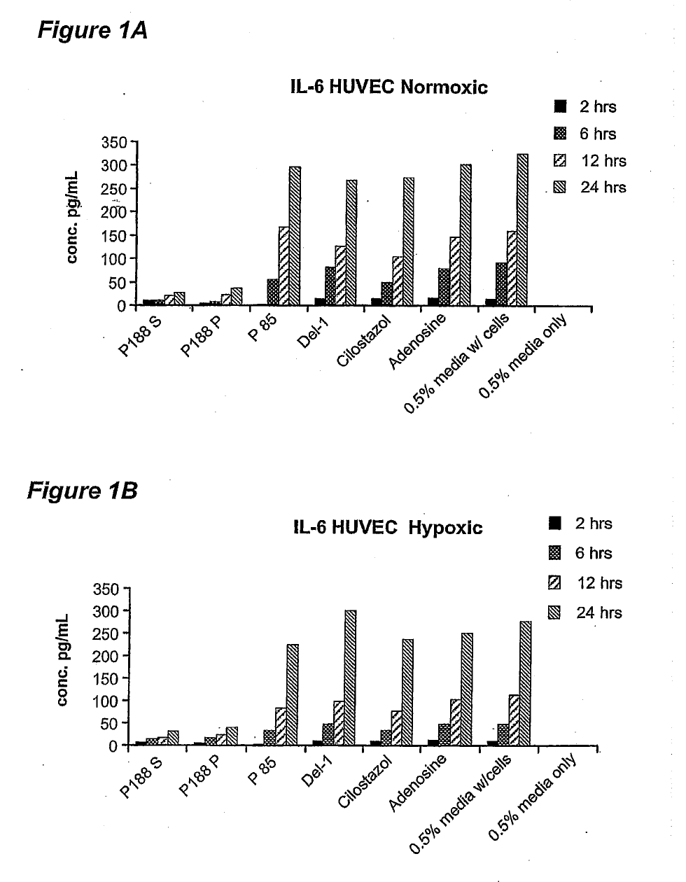

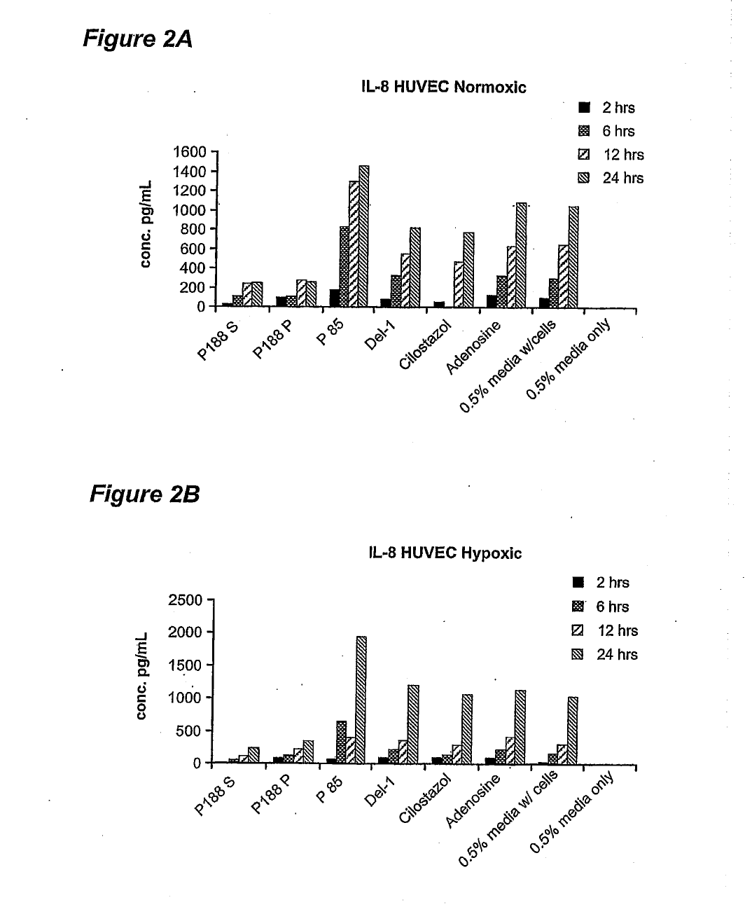

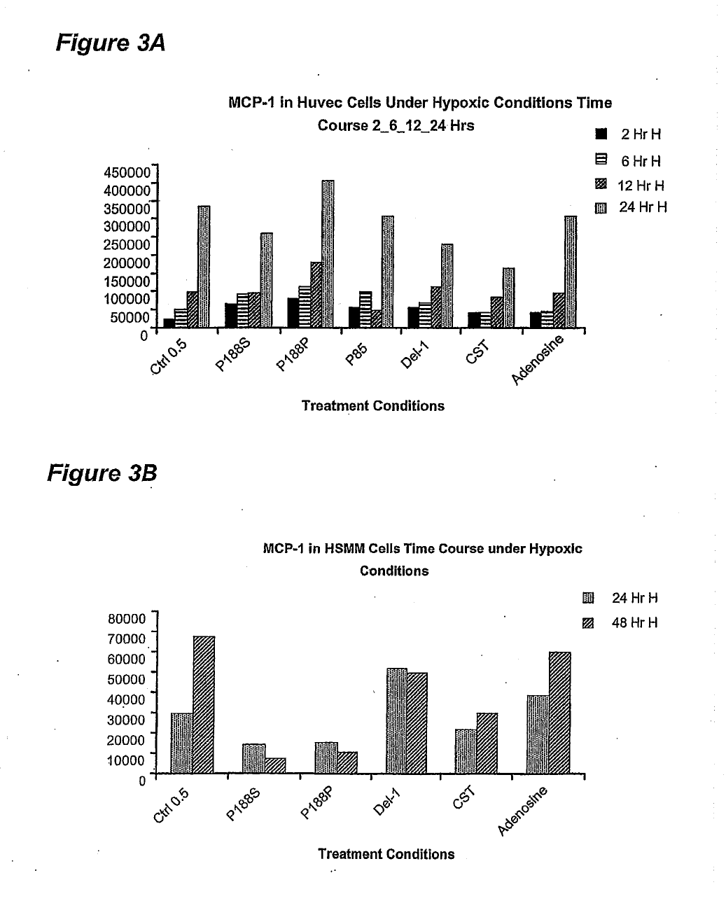

The qualitative results from the protein macroarrays were confirmed by the quantitative ELISAs that used different monoclonal antibodies for detection than the macroarrays, thus increasing confidence in the results. As shown in FIGS. 1A and 1B, HUVEC cells that were untreated or treated with Del-1, CST or adenosine produced similar levels of IL-6 when sampled at various time points in culture under normoxic (1A) and hypoxic (1B) conditions. Poloxamer-188 treatment of HUVECs resulted in decreased levels of IL-6 released into the supernatant over the 24 hours of incubation under both normoxic and hypoxic conditions. Similarly, poloxamer-188 treatment of HUVECs resulted in decreased levels of IL-8 released into the supernatant over the 24 hours of incubation under both normoxic and hypoxic conditions as shown in FIGS. 2A and 2B. The difference between treatment groups and controls was not apparent for MCP-1, with the exception of poloxamer-235 treatment which resulted in slightly lower levels of MCP-1 into the medium than any of the other treatment or control groups as shown in FIG. 3A. Most significantly of these results, poloxamer-235 increased IL-6 and IL-8 levels while poloxamer-188 dramatically decreased the production of IL-6 and IL-8.

### Effects of Various Treatments in Human Skeletal Muscle Myoblast Cells

Myoblast cells did not appear to produce appreciable levels of IL-6 or IL-8, regardless of the treatment of incubation conditions. The IL-6 levels were at the threshold level of detection for the assay system. As with the macroarray analysis, MCP-1 release was highest for all treatments, other than poloxamer-188, during the latter sampling times for both normoxic and hypoxic conditions. Of particular interest, although poloxamer-188 had little differential effect versus other treatment in HUVEC cells, in HSMM cells poloxamer-188 treatment dramatically reduced MCP-1 production under both normoxic and hypoxic conditions as shown in macroarray data presented in FIGS. 3A and 3B. This result was obtained in both the macroarray assay and in the capture ELISA.

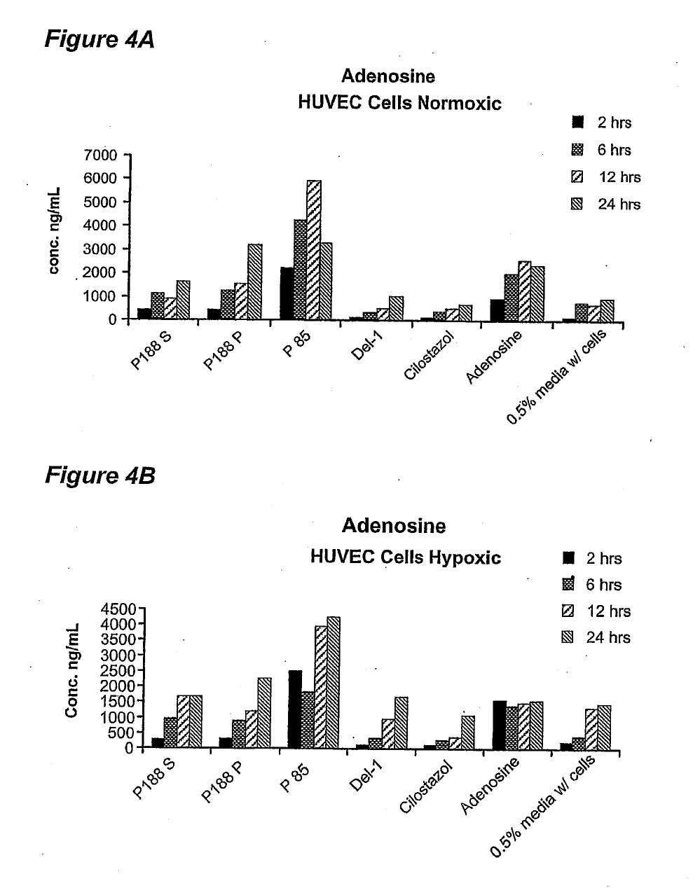

Differential Adenosine Responses between P85 and P188: Confirming results reported in the literature, poloxamer-235 did lead to the higher levels of adenosine in the supernatant versus the other treatments. This increased level of adenosine was seen over time in normoxic cells exposed to poloxamer-235 as shown in FIG. 4A. Cilostazol and Del-1 protein appeared the least stimulatory in adenosine production in these studies, while poloxamer-188 trended toward an intermediate level of release into the medium. Similar results were obtained in hypoxic cells.

Delivery schemes for treatment of inflammation. In one embodiment, inflammation mediated by IL-6 and/or IL-8 is controlled in inflammatory sites by local administration of poloxamer 188 for deposition in an extravascular tissue by intramuscular, intravascular and/or intracapsular injection. By depositing the polymer in an extravascular compartment, the half-life and effective presence of the polymer in the body is greatly extended such that a prolonged effect can be obtained. Local intramuscular administration can be effected by direct injection into the muscle or by a vascular approach where the formulation is introduced into a local isolated portion of the vascular tree that perfuses the affected tissue and is extravasated from the vasculature by pressure into the musculature.

In another embodiment, methods and compounds for treatment of inflammation in coronary arterial disease is provided that includes local intramyocardial administration of a formulation comprising a non-ionic polymer. Local intramyocardial administration can be effected by direct injection into the muscle or by a vascular approach where the formulation is introduced into a local isolated portion of the vascular tree that perfuses the affected myocardium and is extravasated from the vasculature by pressure into the musculature. As with PVD, the compounds can be delivered by “retrograde infusion” or “retrograde perfusion” by which is meant intravenous administration against the path of normal blood flow. For retrograde infusion or perfusion of the heart, a balloon occlusion catheter is passed transvenously into the coronary sinus. From the coronary sinus the catheter can be further advanced into a tributary of the sinus including the great cardiac vein (GCV), middle cardiac vein (MCV), posterior vein of the left ventricle (PVLV), anterior interventricular vein (AIV), or any of their side branches. This delivery modality was originally described for delivery of drugs, cardioprotective agents or cardioplegia during myocardial surgery. (Kar et al. Heart Lung 21 (1992) 148-59; Herity et al. Catheter Cardiovasc Intery 51 (2000) 358-63). Retrograde delivery of naked plasmid DNA encoding the marker proteins LacZ and luciferase was described by Wolff in WO00/15285. Retrograde delivery of plasmid DNA formulated with a non-ionic polymer was described in Valentis WO02/061040.

The anti-inflammatory effects of extravascular polymer deposition may be combined with one or more further agents that are able to stimulate the growth and maturation of new collateral vessels in an ischemic tissue. By agents, it is meant small molecule stimulants as well as biological factors, including proteins and the genes that encode them. Agents involved in angiogenesis may act directly, such as endothelial cell growth factors, or may act indirectly such as through the recruitment of cells involved in the growth of new vessels or through the stimulation of intracellular signaling cascades.

Known biological angiogenic factors include for example Angiogenin, Angiopoietins and Angiopoietin-Like factors, Del-1, E26 Transformation Specific Factors (ETS 1 and 2), Epidermal Growth Factor (EGF), Erythropoietin (EPO), Fibrin fragment E, Fibroblast growth factors: acidic (aFGF) and basic (bFGF), Follistatin, Granulocyte colony-stimulating factor (G-CSF), Hepatocyte growth factor (HGF)/scatter factor (SF), Insulin-Like Growth Factors 1A and 2, Interleukin-8 (IL-8), Kerotinocyte Growth Factor (FGF7), Leptin, Midkine, Nerve Growth Factor Beta, Neuropeptide Y, Placental growth factor, Platelet-derived endothelial cell growth factor (PD-ECGF), Platelet-derived growth factor-BB (PDGF-BB), Pleiotrophin (PTN), Progranulin, Proliferin, Stromal Derived Factor-1 (SDF-1), Transforming growth factor-alpha (TGF-alpha), Transforming growth factor-beta (TGF-beta), Tumor necrosis factor-alpha (TNF-alpha), Vascular endothelial growth factor (VEGF)/vascular permeability factor (VPF) and Vascular Early Response Gene (VERGE). The factors may be provided as recombinant or isolated proteins or as the genes encoding them.

Where further agents are added to a polymer formulation, it is contemplated that the agents function acutely to stimulate angiogenesis or to initiate an angiogenesis cascade while the polymer remains tissue resident and continues to stimulate angiogenesis for a more prolonged period thus resulting in continued improvement and a long term benefit from each administration.

Poloxamer Formulations: The term “block co-polymer” means a polymer composed of two or more different polymers (“co-polymer”) arranged in segments or “blocks” of each constituent polymer. Both poloxamers and poloxamines are block copolymers. The term “poloxamer” means any di- or tri-block copolymer composed of polypropylene oxide and polyethylene oxide. Polypropylene oxide (POP or polyoxypropylene, has the formula (C₃H₆O)ₓ, thus a subunit mw of 58) is a hydrophobe. Polyethylene oxide (POE or polyoxyethylene has the formula (C₂H₄O)ₓ, thus a subunit mw of 44) and is a hydrophile. The common chemical name for poloxamers is polyoxypropylene-polyoxyethylene block copolymer. The CAS number is 9003-11-6. The poloxamers vary in total molecular weight, polyoxypropylene to polyoxyethylene ratio, surfactant properties and physical form in undiluted solution. Physical forms include Liquids (L), Pastes (P) and Flakable solids (F), determined largely by the relative percentage of hydrophobic versus hydrophilic components.

Pluronic® is a trademark for poloxamers manufactured by BASF. In Europe the pharmaceutical grade poloxamers manufactured by BASF is sold under the mark Lutrol. Poloxamers are tri-block copolymers in which the hydrophobe propylene oxide (PO or PPO) block is sandwiched between two hydrophile ethylene oxide (PE or PEO) blocks, in accordance with the following general formula and structure of FIG. 2. Reverse poloxamers (such as BASF “reverse Pluronic®s”) have a central EO (aka PEO) moiety sandwiched between two PO (aka PPO) moieties with the following general formula and structure of FIG. 6.

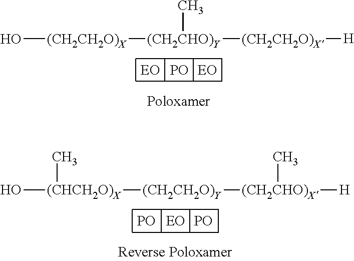

In the nomenclature of poloxamers, the non-proprietary name “poloxamer” is followed by a number, the first two digits of which, when multiplied by 100, equals the approximate molecular weight (“mw”) of the polyoxypropylene (“POP”) and the third digit, when multiplied by 10 equals the approximate % by weight of the polyoxyethylene (“POE”). Thus, poloxamer 188 would have an average POP mw of approximately 1800 and an average POE % of 80%. Calculated according to the poloxamer nomenclature for poloxamer 188 (a.k.a. F68) the average number of POP groups are derived as follows: 1800÷58 (mw of C₃H₆O)=31 POP units. The total mw=1800÷(20/100)=9000. The average number of POE are derived as follows: (total approximate mw−mw POP)÷44 (mw of C₂H₄O) is thus (9000−1800)=7200÷44=163. Therefore the formula for poloxamer 188 (a.k.a. F68): HO—(C₂H₄O)₈₂—(C₃H₆O)₃₁—(C₂H₄O)₈₂—H.

Alternatively, from the formula HO—(C₂H₄O)ₓ—(C₃H₆O)_{y}—(C₂H₄O)ₓ—H, the average molecular weight, the percentage of POE, and the numbers of POE and POP units can be otherwise derived depending on the variable known. Thus if the total mw and % POE is known the formula can be derived as follows:

Average number of POE groups are derived as follows: (total approximate mw−18 (the mw of the terminal hydroxy and hydrogen groups))×wt % POE)=mw POE÷44=number of POE groups, therefore for F68; ((8400−18)×80%)=6705.6÷44=152.4(÷2=76).

Average number of POP groups can be derived as follows: ((total approximate mw−18)−mw POE)=mw POP÷58. Therefore for F68: ((8400−18)−6705.6=1676.4÷58=30. The formula for poloxamer 188, a.k.a. F68, would thus be: HO—(C2H₄O)₇₆—(C₃H₆O)₃₀—(C₂H₄O)₇₆—H.

In the BASF nomenclature, a letter describing the physical form of the poloxamer (whether Liquid “L”, Paste “P” or Flakable “F”) is followed by a first number arbitrarily representing the molecular weight of the POP step-wise up the y axis of the poloxamer grid and the second number representing the % POE. PLURONIC® F68 is the BASF trademark for poloxamer 188. BASF gives 84-00 as the average mw for F68 but states an average mw of 8600 for F68NF grade and gives values of POE=80 (×2), and POP=27: therefore the POP mw=1566, POE %=81.6% with the resulting formula: HO—(C₂H₄O)₈₀—(C₃H₆O)₂₇—(C₂H₄O)₈₀—H which would have a resulting mw of 18+7040+1566=8624. Commercially available N.F. grade F68 obtained from either BASF or Spectrum Chemicals has an average molecular weight range of 7,680-9,510 Da with a weight percent polyoxyethylene of 81.8±1.9% and an unsaturation fraction of 0.026±0.008 mEq/g. The molecular weight of the polyoxypropylene component is 1750.

Because in actual practice, poloxamers are typically synthesized according to a process in which a hydrophobe of the desired molecular weight is generated by the controlled addition of propylene oxide to the two hydroxyl groups of propylene glycol followed by addition of ethylene oxide to sandwich the hydrophobe between hydrophilic groups results in a population of molecules in a relatively circumscribed range of a molecular weights characterized by a hydrophobe having a defined average molecular weight and total average percentage of hydrophile groups. For example, commercially available USP/NF grade F68 obtained from either BASF (LUTROL® F68, CAS No: 9003-11-6) or Spectrum Chemicals has an average molecular weight range of 7,680-9,510 Da with a weight percent polyoxyethylene of 81.8±1.9% and an unsaturation fraction of 0.026±0.008 mEq/g. The molecular weight of the polyoxypropylene component is 1750.

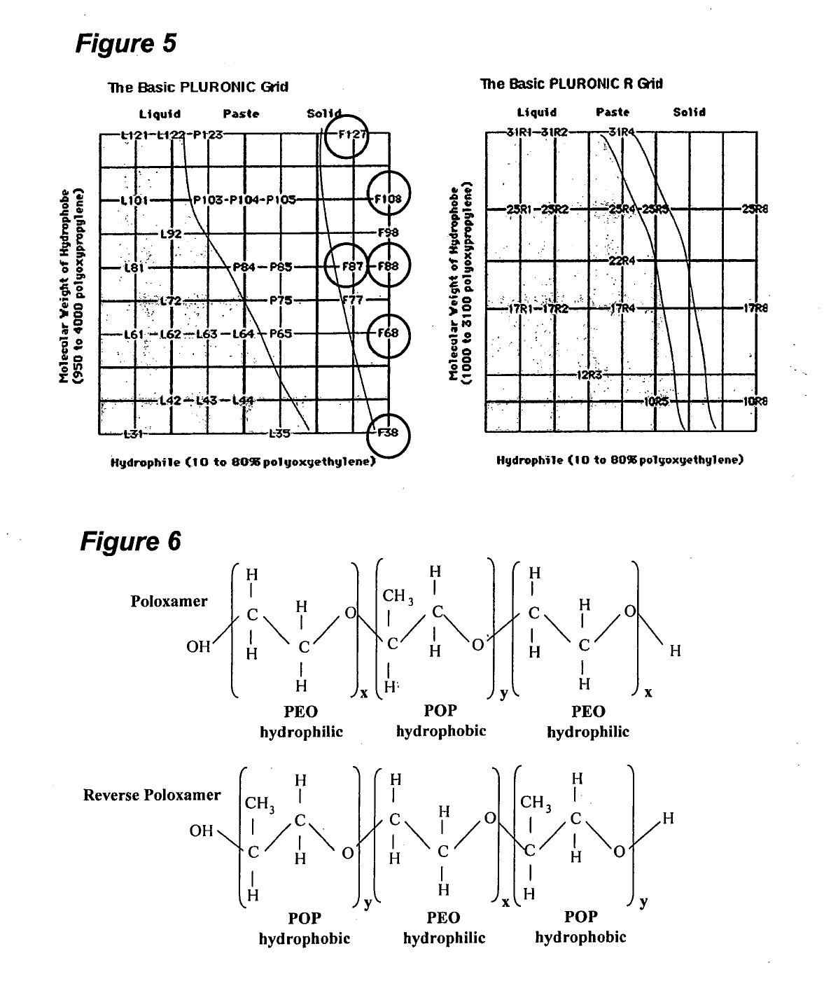

Since both the ratio and weights of EO and PO vary within this family of surfactants, BASF developed a PLURONIC® grid to provide a graphic representation of the relationship between copolymer structure, physical form and surfactant characteristics as reproduced in FIG. 5. On the PLURONIC® surfactant grid the molecular weight ranges of the hydrophobe (propylene oxide) are plotted against the weight-percent of the hydrophile (ethylene oxide) present in each molecule. Poloxamer species defined by their location on the PLURONIC® grid can be expected to have shared properties that are a function of their total molecular weight and relative hydrophobicity. As used herein, the phrase “having the characteristics of” a particular poloxamer means those poloxamers that exhibit copolymer structure, physical form and surfactant characteristics similar to those of the named poloxamer.

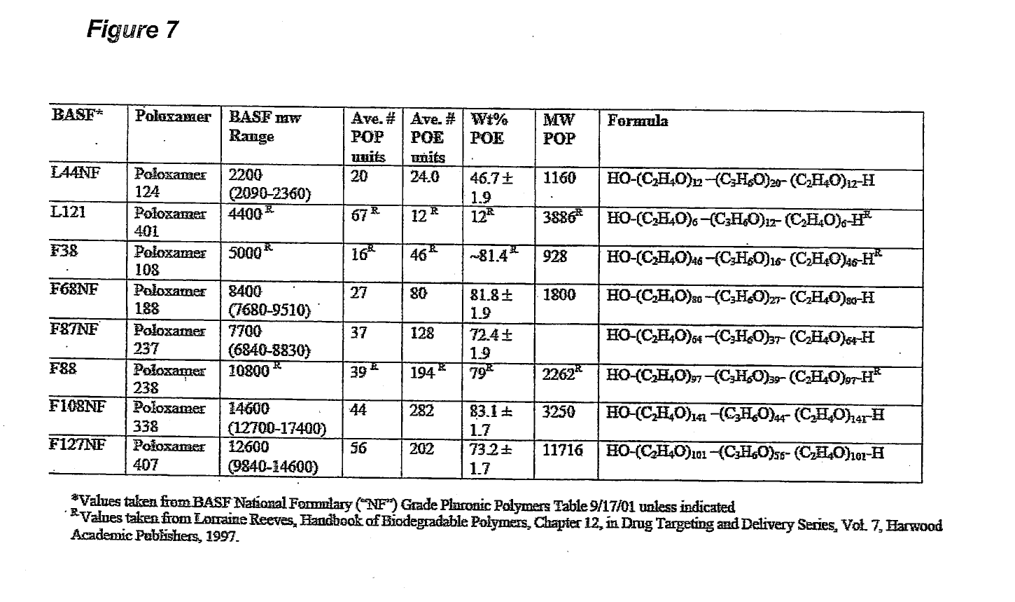

The PLURONIC® Grid, a facsimile of which is shown on FIG. 5, clarifies the use of the letter-number combinations to identify the various products of the PLURONIC® series. The alphabetical designation explains the physical form of the product: “L” for liquids, “P” for pastes, “F” for solid forms. The first digit (two digits in a three-digit number) in the numerical designation, multiplied by 300, indicates the approximate molecular weight of the hydrophobe (vertical axis at the left of the Grid). The last digit, when multiplied by 10, indicates the approximate ethylene oxide content in the molecule, read from the horizontal axis. FIG. 7 sets out the molecular weight range, percentage of co-polymer constituents and approximate formula of several poloxamers by both generic (poloxamer) and corresponding BASF tradenames.

As used herein, the term “poloxamine” refers to poly(oxyethylene)-poly(oxypropylene) (POE-POP) block copolymers where a POE-POP unit is linked to another POE-POP unit by an amine and having the general structure (POEₙ-POPₘ)₂—N—C₂H₄—N—(POPₘ—POEₙ)₂. TETRONIC® and TETRONIC R nonionic surfactants produced by BASF are exemplary poloxamines. By virtue of their amine group, poloxamines may have a positive charge if unprotonated but are not thought to have sufficient charge to condense negatively charged DNA for example and are thus included within the group of non-ionic polymers for purposes of the present invention.

Poloxamines are in the alkoxylated amine chemical family and have a slightly different chemical structure. The hydrophobic center consists of two tertiary amino groups carrying both two hydrophobic PPO chains of equal length each followed by a hydrophilic PEO chain. Poloxamines can still be described as a tri-block copolymer although bulkier than poloxamers. Poloxamines of the BASF Tetronic® type have the chemical name: 1,2-Ethanediamine, polymer with the following formula: (POEₙ—POPₘ)₂—N—C₂H₄—N—(POPₘ—POEₙ)₂ and the CAS number: 11111-34-5. Reverse Tetronics® have the formula (POPₙ—POEₘ)₂—N—C₂H₄—N—(POEₘ—POPₙ)₂ and the CAS number: 26316-40-5.

Poloxamers are relatively non-toxic surface active compounds that have long been used as food additives, defoamers, antistatic agents, demulsifiers, detergents, wetting agents, gelling agents, emulsifiers, dispersants and dye levelers. (See Merck Index, 12^{th} Ed. Compound 7722. Poloxamers). In pharmacological applications, poloxamers are used as dispersing and wetting agents for oral, topical and parenteral formulations (See BASF Lutrol® F69 Technical Information January 2004 “Poloxamer 188 for the pharmaceutical industry.”). Used as excipients in the above examples, poloxamers have not been considered to be active ingredients.

Use of ethylene oxide and propylene oxide copolymers to treat an embolus or a thrombus has been described (See U.S. Pat. No. 3,641,240). The use of poloxamers, especially poloxamer-188, by intravenous injection, either alone or in combination with other compounds, including but not limited to for facilitating blood flow in the treatment of various hematological disorders is the subject of a number of patents granted to Robert Hunter. (See e.g. U.S. Pat. Nos. 4,897,263 and 5,089,260). The concept behind all of these inventions is that surface active poloxamers in “effective amounts” may improve blood flow by reducing pathological hydrophobic interactions including adhesion of macromolecules and cells in the microvasculature and coronary vascular resistance.

Poloxamers are not metabolized and are reported to be quickly eliminated from the blood with an estimated half-life of approximately two hours. (See U.S. Pat. RE No. 36,665). Stated applications thus involve acute interventions by intraveneous poloxamer administration including for treatment of myocardial damage in reperfusion, preservation of organs for transplantation, treatment of sickle cell crisis, and in invasive procedures for removing blockages in vessels including balloon angioplasty where blood flow is stated to be reduced by hydrophobic interactions. (See e.g. U.S. Pat. No. 5,030,448).

Phase II clinical trials were undertaken by Burroughs Wellcome and Cytrx to determine the ability of GMP grade poloxamer-188 (trade named RheothRX) to reduce the number of heart attacks, especially a second attack that might follow shortly after the first. An initial 45 patients enrolled were randomized to receive placebo or a low-dose regimen of poloxamer-188 (150 mg/kg/h over 1 hour and then 15 mg/kg/h over 47 hours). This computes to a loading dose of 10.5 grams for a 70 kg patient, followed by a further 49 grams for a total dose of 60 grams of poloxamer. Once this dose was determined to be safe by a safety committee, the final 69 patients received placebo or a high-dose poloxamer-188 regimen (300 mg/kg/h over 1 hour and then 30 mg/kg/h over 47 hours). This computes to a loading dose of 21 grams for a 70 kg patient, followed by a further 98.7 grams for a total dose of 120 grams of poloxamer. A 48-hour infusion of poloxamer 188 was chosen because prior work in a canine model of 90 minutes of coronary occlusion and 72 hours of reperfusion demonstrated superior reduction in myocardial infarct size with a 48-hour poloxamer 188 infusion compared with a 4-hour infusion or a saline placebo. Schaer et al. Circulation 94 (1996) 298.

In Phase III clinical trials, patients were randomized to a control group (n=963) or to receive RheothRx. Patients receiving RheothRx were allocated to receive a 1-hour bolus only (regimen A, n=844), an additional 11-hour infusion at a low dose (target serum concentration of 0.5 mg/mL) (regimen Y, n=490), or an additional 23-hour infusion at a low dose (regimen B, n=483). Three higher doses (1-hour bolus+low-dose infusion for 47 hours, 1-hour bolus+high dose, target serum concentration of 1.0 mg/ml for 24 hours, or 1-hour bolus+high dose for 48 hours) were discontinued because of high rates of renal dysfunction (8.8%). Renal dysfunction was also observed at lower doses (regimen A, 3.1%; Y, 2.7%; and B, 4.1%) compared with the control patients (1.0%). There was no significant difference in the composite outcome of death, cardiogenic shock, or reinfarction at 35 days (all RheothRx, 13.6%; control, 12.7%). Collectively, analysis of the data in almost 3000 patients showed that RheothRx had no effect on mortality rates although was associated with renal toxicity in some patients. Circulation. 96 (1997) 192.

In the Phase III trial, a transient elevation in creatinine was noted in elderly patients with pre-existing renal disease. The reversible renal toxicity noted in the Burroughs Wellcome trial caused CytRx to investigate the cause of the toxicity before proceeding with a Phase III trial of this high IV dose for the treatment of sickle cell crisis. Preclinical work indicated the toxicity was due to the low molecular weight fraction of poloxamer-188 and a “purification” process was thus developed to eliminate both this fraction and a high molecular weight fraction. An animal model of renal disease indicated that the product without the low and high molecular weight fractions did not induce toxicity although the relevance to humans is unknown. The name of the CytRx “purified” product is Flocor™, stated to be useful in enhancing microvascular blood flow, inhibiting inflammation and enhancing thrombolysis in the presence of lytic agents.

In a further clinical trial with RheothRX for treatment of sickle cell crisis, CytRx took purified poloxamer-188 through Phase III development in about 127 patients. As published in the Journal of the American Medical Association Vol 286 No. 17 (Nov. 7, 2001) 2099-2106, the poloxamer was formulated at a concentration of 150 mg/ml (15%) in buffered saline and the dose given intravenously was 100 mg/kg for 1 hour followed by 30 mg/kg for 47 hours. For a 70 kg patient, this is a loading dose of 7 grams followed by a further 98.7 grams for a total dose of 105.7 grams of purified poloxamer.

In contrast, in preferred embodiments of the present invention, extravascular depot delivery by multiple intramuscular injections is provided but with a considerably lower acute total body dose than that used in the aforementioned trials. For example, where 12-42 injections at 2 ml per injection are given at a poloxamer concentration from 1 to 6%, the total dose low end dose would be 12 injections×2 ml/inj×10 mg/ml (1%)=240 mgs or 0.24 g total. The total high end dose of this range is calculated as 42 injections×2 ml/inj×60 mg/ml (6%)=5.04 grams total. If a concentration of 15% is utilized, the calculated dose is 42 inj×2 ml/inj×150 mg/ml=12.6 grams total. The relative amounts of low molecular weight components hypothesized to cause toxicity by acute injection in the Cytrx trial are calculated as follows: 0.24 grams=0.0082 grams of low molecular weight material; 5.04 grams=0.174 grams of low molecular weight material; and 12.6 grams=0.428 grams of low molecular weight material. In comparison, given the high volume of material delivered in the Cytrx trial, even with purified material 0.233 grams of the low molecular weight component would have been present even in the lowest dose of 21.2 grams in the Cytrx trial using purified poloxamer.

Poloxamers including poloxamer-188 have been investigated for enhancing wound repairs by sealing of cell membranes after injury including by electroporation (Lee R C, et al. Proc. Natl. Acad. Sci. (1992) 89(10): 4524-4528), heat shock (Padanilam J T, et al. Annals of NYAS, (1994) Vol. 720, pp. 111-123), and neurotoxins (Marks J D, et al., Soc Neurosci Abs 24(1): 462, 1998.).

In high concentrations, certain poloxamers form a polymer hydrogel. Such hydrogels have been tested for drug delivery and sustained release. Poloxamer gel formulations have been used for delivery of genes to the vascular tissue in vivo using viral vectors, where the gel was expected to restrict movement of the viral formulation from the site of administration. (Feldman et al. Gene Therapy (1997) 4, 189-198; Van Belle et al. Human Gene Therapy (1998) 9, 1013-1024; Hammond et al., U.S. Pat. No. 6,100,242).

The use of non-gel forming concentration of hydrophilic type poloxamers for DNA delivery was disclosed in Cytrx WO95/10265. Cationic or positively charged poloxamers have been developed that form ionic interactions with negatively charged DNA molecules and thus condense the DNA into particles for gene delivery. (See e.g. Kabanov et al U.S. Pat. No. 5,656,611 and U.S. Pat. No. 6,353,055). The use of hydrophilic type poloxamers at non-gel forming concentrations for delivery of nucleic acids to muscle was taught in Valentis WO01/65911.

FIG. 5 shows the chemical characteristics of poloxamers determined to increase delivery of plasmid DNA to muscle. Effective “F” group of poloxamers are circled on FIG. 5 and include poloxamers represented by poloxamer-108 (PLURONIC® F38), poloxamer-188 (PLURONIC® F68), poloxamer-237 (PLURONIC® F87), poloxamer-238 (PLURONIC® F88), poloxamer-338 (PLURONIC® F108NF) and poloxamer-407 (PLURONIC® F127). Liquid form poloxamers-124 (PLURONIC® L44NF) and poloxamer-401 (PLURONIC® L121) were also found to increase gene expression. (See Valentis WO01/65911 and WO02/061040). In particular, these poloxamers have been shown by Valentis to significantly increase the delivery of plasmid DNA with concomitant expression of angiogenic transgenes in both skeletal and cardiac muscle.

In light of the present discovery, this effect may now be explained in part by an activity in increasing vascularity sufficient to induce angiogenesis in the absence of added angiogenic biological agents, and/or to promote to continued improvement. The present inventors have now surprisingly found that poloxamers are themselves able to ameliorate symptoms of intermediate claudication when administered into the muscle in an affected limb of PAD patients and can effect long term improvements in peak walking time (PWT) and ankle brachial index (ABI). The present inventors have also surprisingly found that poloxamer-188 in particular has the property of selectively decreasing the production of the inflammatory cytokines IL-6 and IL-8 in endothelial cells and myoblasts. Poloxamer-188 further has a specific effect of decreasing the production of MCP-1 in myoblast cells.

Preclinical Studies: The preclinical pharmacology of human Del-1 plasmid versus empty plasmid formulated with poloxamer was evaluated in mouse and rabbit animal models. These plasmids were formulated with the same non-ionic polymer, 5% poloxamer-188, in aqueous solution. The effect of formulated hDel-1 plasmid on capillary:myofiber ratio in normoxic muscle of CD-1 mice 7 days post injection showed that a single intramuscular (IM) 10 μg dose of formulated hDel-1 plasmid increased capillary:myofiber ratio by approximately 60% (p<0.01). Comparable effects were observed using human and murine formulated Del-1 plasmids. This result did not suggest a significant effect attributable to the poloxamer.

The effects of formulated hDel-1 plasmid versus formulated VEGF165 plasmid and empty plasmid were investigated in a murine hindlimb ischemia model. Bilateral ischemia was induced in hindlimbs of CD-1 mice by ligation of the femoral artery. A control group underwent sham surgery without femoral artery ligation. Immediately following ligation of the femoral artery, mice were treated with IM injections of 70 μg of formulated hDel-1 plasmid per hindlimb divided among the tibialis anterior (10 μg), gastrocnemius (20 μg), and quadriceps (40 μg) muscles. Formulated hVEGF165 plasmid was included for comparison since studies have suggested that overexpression of VEGF may lead to increased collateral formation in ischemic tissue. Exercise tolerance was then determined at weekly intervals through four weeks post surgery. The effects of formulated hDel-1 plasmid were not different from VEGF although both formulated hDel-1 and hVEGF₁₆₅ plasmids increased exercise tolerance versus formulated control plasmid (p<0.05). This result did not suggest a significant effect attributable to the poloxamer.

A study using a surgical hindlimb model was also conducted in New Zealand rabbits. Poloxamer formulated hDel-1, VEGF, or control plasmid was injected into the medial thigh of New Zealand rabbits 3-4 days after surgical excision of the femoral artery (5 mg plasmid dose divided among 10 injection sites, 0.5 mL/site). Angiography was performed immediately after surgery and again at one month. Results for the number of new collateral vessels crossing over the mid thigh region showed that formulated hDel-1 and VEGF plasmid elicited a greater than two-fold increase in collateral vessel development over the one-month course of the experiment (p<0.01) compared with empty plasmid. This result did not suggest a significant effect attributable to the poloxamer.

Therapy in Human PAD: Atherosclerosis is the most common cause of chronic arterial occlusive disease of the lower extremities and can lead to clinical conditions ranging from intermittent claudication (ischemic pain) to ulceration and gangrene. The arterial narrowing or obstruction that occurs as a result of the atherosclerotic process reduces blood flow and tissue perfusion to the lower limb during exercise or at rest. A spectrum of symptoms results, the severity of which depends on the extent of the involvement and the available collateral circulation. The superficial femoral and popliteal arteries are the vessels most commonly affected by the atherosclerotic process. The distal aorta and its bifurcation into the two iliac arteries are the next most frequent sites of involvement.

PAD accounts for a sizable portion of annual health-care expenditures. Furthermore, beyond the actual health-care dollars spent, PAD is a major cause of disability, loss of work/wages, and lifestyle limitations (Rosenfield K, and Isner J M (1998). In: Comprehensive Cardiology Medicine. J Topol, ed. Lippincott-Raven Publishers, Philadelphia 3109-3134.) It has been estimated. that PAD affects 1 in 20 people over the age of 50 or approximately 8 to 12 million people in the United States, being more commonly diagnosed in men than in women (Creager, M A. Cardiol Rev. 9 (2001) 238-245). Regardless of the location and distribution of PAD within the lower extremity vasculature, claudication symptoms are most frequently localized to the muscles of the calf and are manifested as alteration in resting hemodynamic measurements in the lower extremity. Patients with IC generally have an ABI between 0.4 and 0.9, with lower values being associated with increasing disease severity and cardiovascular risk. (Greenland P, et al. Circulation (2000) 101:E16-22). As blood vessel narrowing increases, critical ischemia (CLI) can develop when the blood flow does not meet the metabolic demands of tissue at rest. It is manifested by rest pain, non-healing ulcers and gangrene and may lead to amputation.

The principles for the treatment and management of patients with IC and/or PAD have been the subject of several recent reviews and scientific statements. (See e.g. Weitz J I, et al. Circulation (1996) 94:3026-49; Hiatt W R. N Engl I Med (2001) 344:1608-21). Most patients are treated primarily to relieve lower extremity symptoms, increase functional walking capacity and quality of life, prevent the progression of disease, and preserve limb tissue. Management of risk factors, lifestyle interventions, and pharmacologic treatment with agents to provide symptomatic relief have a central role in improving function and quality of life and retarding the progression to advanced endpoints such as the rest pain, nonhealing ulcers, gangrene and cardiac death. Smoking cessation, institution of antiplatelet therapy, and ability to institute statin therapy represent important goals in the treatment of the patients with IC. In individuals with severe symptoms and identifiable proximal inflow disease, surgical or percutaneous revascularization for aortoiliac disease may provide durable treatment. Infrainguinal disease, even if extensive, very rarely justifies surgical intervention for claudication. Although select patients with superficial femoral artery disease and claudication may be considered for surgical treatment or percutaneous recanalization, these techniques are not successful in the vast majority. Similarly in patients with distal disease afflicting the tibio-peroneal circulation, there is a limited role for primary infrapopliteal angioplasty or surgery unless the patient is experiencing critical limb ischemia. Thus, the treatment of infrainguinal disease is predominantly medical in patients with IC.

A human Phase I clinical trial was conducted to test the safety of a formulation (VLTS-589) including of 1 mg/ml plasmid encoding the angiogenic protein Del-1 in an aqueous saline solution of the facilitating agent poloxamer-188, National Formulary [NT], 50 mg/ml and the excipients 0.28 mg/ml Tris-(hydroxymethyl)-aminomethane, United States Pharmacopoeia (USP) (Tris, USP), and 0.44 mg/ml Tris-(hydroxymethyl)-aminomethane hydrochloride (Tris-HCl). For manufacture, the drug substance (Del-1 plasmid) and facilitating agent (poloxamer) were aseptically mixed using an in-line mixing process and terminally sterile filtered using a 0.2-μm absolute filter. Vials were filled and lyophilized under aseptic conditions. Following lyophilization, the drug product was stored at 2° C. to 8° C. VLTS-589 was supplied as a white to slightly yellow, sterile, lyophilized powder in sterile 15-mL glass vials, stoppered with 20-mm gray stoppers, and sealed with aluminum flip-off caps.

Poloxarrier was considered a facilitating agent because it “facilitates” the increased expression of Del-1 protein from the Del-1 encoding plasmid that was administered as part of the formulation. The Tris, Tris-HCl and saline were considered pharmaceutically acceptable excipients. As used herein the term “excipient” means an ingredient intentionally added to a therapeutic product which is not intended to exert a therapeutic effect at the intended dosage although they may act to improve product delivery and biocompatibility by adjusting characteristics such as pH and/or tonicity. Many other suitable excipients are known to those of skill in the pharmaceutical arts.

In the clinical trial, poloxamer-188 having the approximate calculated chemical composition was used: HO(CH₂CH₂O)₈₀(CH(CH₃)CH₂O)₂₇(CH₂CH₂O)₈₀H. The drug product was lyophilized until use. For use, the lyophilized drug product (lacking NaCl) was reconstituted with sterile 0.9% sodium chloride for injection.

The trial included 27 patients in a dose escalation protocol where the patients initially exhibited an ABI of ≦0.85. Assessments made prestudy and at 30 and 90 days evaluated exercise tolerance, ABI and vascularity using angiography (pre and 30 days). The formulation was administered in a ring pattern of dose escalation of 3 mg to a total of 84 mg of plasmid DNA by increasing number of injections at a single time of administration. Thus, the first cohort received a single 3 ml injection. The second cohort received 2 injections of 3 ml each. The third cohort received a full ring of 4 injections, each of 3 ml. The fourth cohort received 12 injections in a pattern of 4 injections in each of three rings. The fifth cohort received 20 injections in a pattern of 4 injections in each of 5 rings. The final sixth cohort received 28 injections, 4 injections per ring in each of 7 rings for a total of 84 mg of plasmid DNA administered in a single leg. An additional cohort received the same dose but in a longitudinal track pattern down the posterior aspects of the legs in lieu of the circumferential ring patterns.

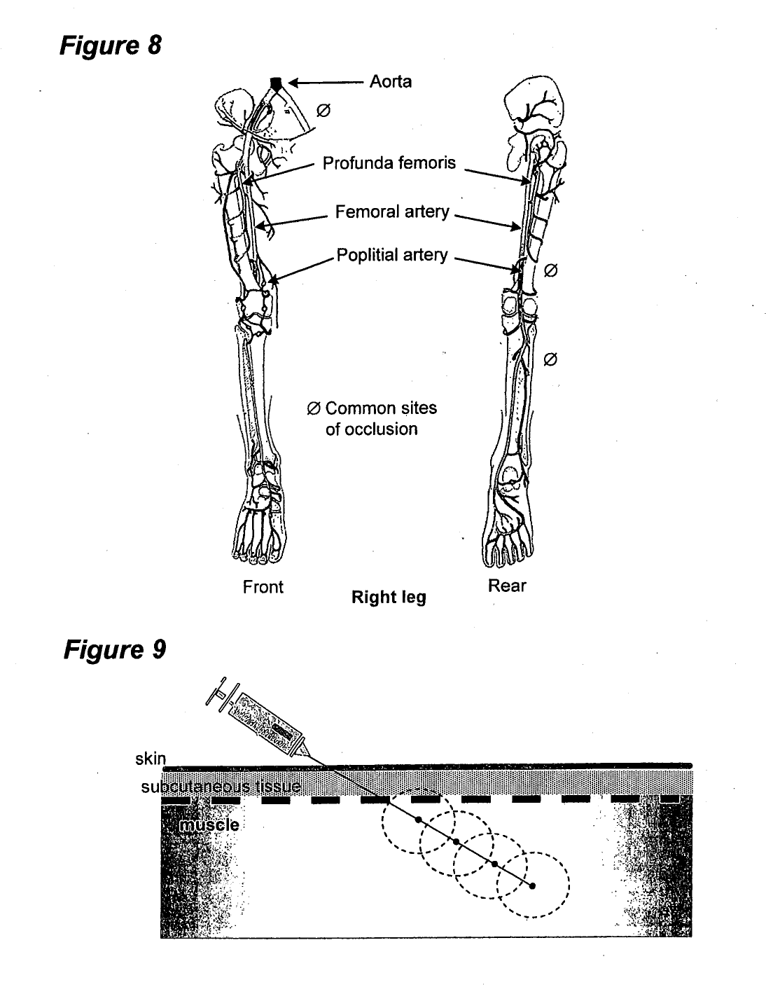

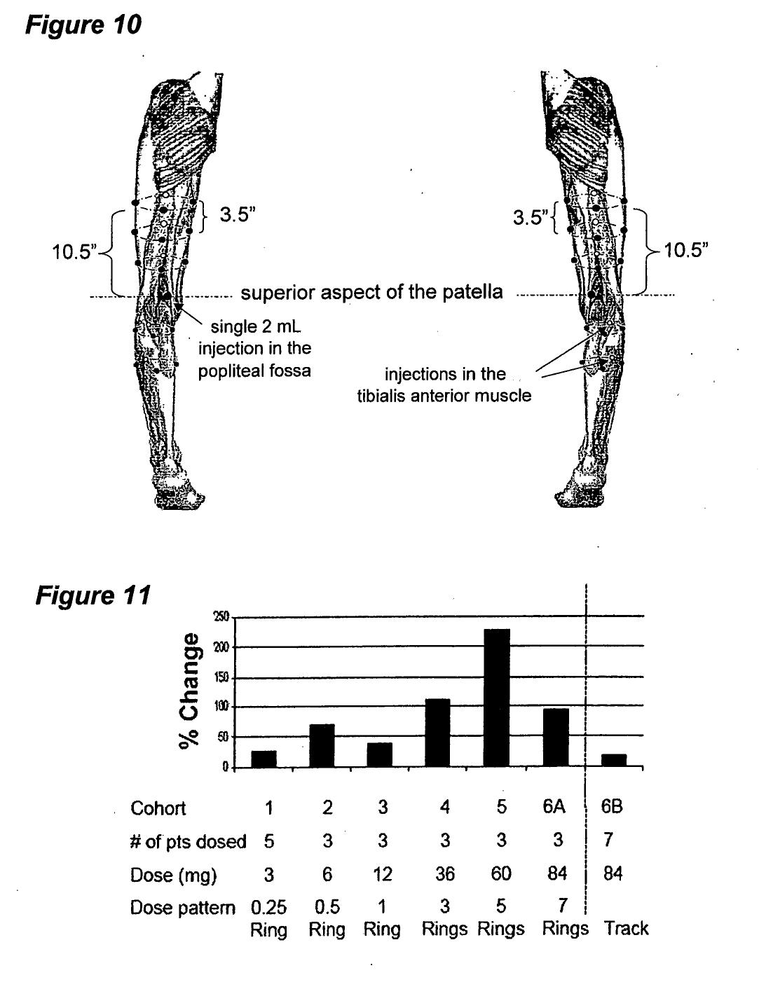

In the case of a leg, formulations that are delivered to the leg in ring pattern beginning near the path taken by the femoral artery and proceeding downward as the femoral artery feeds into the popliteal artery are administered in a “flow to no-flow” where the pattern of deposition sites begins above an area of occlusion of an artery and continues longitudinally down the extremity toward an area of clinically relevant ischemia. The vascular anatomy of the leg is depicted in FIG. 8. Injections are delivered at an angle where a volume in the syringe is gradually pushed out in increments as the needle is removed from the muscle tissue as graphically depicted in FIG. 9. Experience with this method suggests that a 0.5 cc IM injection will treat a sphere of tissue approximately 3 cubic centimeters in volume. FIG. 10 depicts a ring patter of injection in accordance with the invention.

In order to provide a flow to no-flow administration regime in the case of the leg, injections are given both above and below the knee. A trend towards improvement in exercise tolerance at 90 days was noted with escalating dose up to the 5 ring pattern as shown on FIG. 10.

Phase II Trial: Subsequent to the Phase I safety trial, a Phase II double blind “placebo” controlled trial was conducted comparing the poloxamer formulation alone (“placebo”) with the formulation containing plasmid DNA encoding Del-1. A double-blind study is a clinical study of potential and marketed drugs, where neither the investigators nor the subjects know which subjects will be treated with the active principle and which ones will receive a placebo. A placebo is typically defined as an inert substance or dosage form that is identical in appearance, flavor and odor to the active substance or dosage form. Placebos are used as negative controls in bioassays or in clinical studies.

The Phase II, multicenter, double-blind, placebo-controlled trial involved subjects with IC secondary to predominately infrainguinal peripheral arterial disease who received a single treatment of VLTS-589 (84 mg, or 84 mL) or placebo (84 mL) administered as 21 intramuscular (IM) injections of 2 mL each into the index (more symptomatic) lower extremity and 20 injections of 2 mL each into the bilateral lower extremity during one procedure. With the exception of the addition of the active drug substance (Del-1 encoding plasmid), the composition and manufacture of the placebo was identical to that of the drug product (VLTS-589). Placebo was supplied as a white to slightly yellow, sterile, lyophilized powder in sterile 15-mL glass vials, stoppered with 20-mm gray stoppers, and sealed with aluminum flip-off caps.

Clinical Endpoints: The primary endpoint objectives were to: 1) to evaluate the safety and tolerability of IM injections of VLTS-589 compared with placebo, administered bilaterally to the lower extremities, in subjects with intermittent claudication (IC) secondary to predominantly infrainguinal peripheral arterial disease, and 2) to evaluate the change in peak walking time (PWT) from baseline to Day 90 for subjects receiving VLTS-589 compared with subjects receiving placebo. The secondary endpoints were to evaluate the: 1) change in PWT with VLTS-589 from baseline to Days 30, 180 and 365 compared with placebo; 2) percent and absolute change in resting ankle-brachial index with VLTS-589 from baseline to Days 30, 90, 180 and 365 compared with placebo; 3) percent change in the claudication onset time (COT) from baseline to Days 30, 90,180 and 365 compared with placebo; and 4) absolute changes in COT with VLTS-589 from baseline to Days 30, 90, 180 and 365 compared with placebo.

Study Subjects: 100 patients with bilateral disease were enrolled having an ABI of ≦0.8 in both legs and were entered into an equal randomization. Subjects were treated as outpatients during the course of the trial. Subjects were monitored during administration of VLTS-589 for signs of systemic or local treatment-related toxicity. Safety assessments included the reporting of AEs, clinical laboratory evaluations, vital signs measurements, physical examinations, ECGs, and concomitant medications. After all subjects completed the Day 90 visit, an interim analysis was performed on the efficacy and adverse event data.

Ankle-brachial index or toe-brachial index: The ABI is the ratio of the systolic blood pressure at the ankle, divided by the systolic blood pressure in the arm. This is performed after the subject has been lying supine for at least 10 minutes prior to the treadmill test. The ABI is obtained by determining the dorsalis pedis and posterior tibial systolic blood pressures in both ankles and the brachial systolic blood pressures in both arms, using a 5-7 MHz Doppler ultrasound instrument. The ABI for each lower extremity is calculated by dividing the higher of the 2 ankle readings, by the higher of the 2 brachial readings, in each lower extremity. For subjects with an ABI of >1.3 (noncompressible calcified arteries) a toe-brachial index (TBI) in the great toe was allowed. The TBI is the ratio of the systolic blood pressure at the first toe divided by the systolic blood pressure in the arm. In this case, the TBI must be ≦0.7 for subject qualification.

Statistical Methods: Two populations are defined in the analyses: 1) safety population defined as all subjects who received any study drug, and 2) efficacy population consisting of all subjects with at least one post-VLTS-589 or placebo administration. Continuous variables were summarized using the mean, the standard deviation, the median, the minimum value, and the maximum value. Categorical variables were summarized using frequency counts and percentages. Assessment of efficacy was made by comparing efficacy parameters between VLTS-589 and placebo control groups. All comparisons were two-tailed with an α-value of 0.05. The null hypothesis was that there is no difference between VLTS-589 and placebo.

Using the primary endpoint, a sample size of 100 subjects was used for the study. A standard deviation of 2.5 minutes and a clinically significant difference of 1.5 minutes in the change in PWT from baseline to Day 90 were used to calculate the sample size. This standard deviation is based on primary efficacy endpoint in a previous study. Assuming a standard deviation of 2.5 minutes, a 2-sided t-test for independent samples with a significance level of 0.05 would require 45 completed subjects per treatment group in order to have 80% statistical power to detect a difference of at least 1.5 minutes between VLTS-589 arm and placebo (nQuery Advisor, Version 4.0). By comparison, the observed difference was 1.17 minutes in the TRAFFIC study (Lederman R J et al. Therapeutic angiogenesis with recombinant fibroblast growth factor-2 for intermittent claudication (the TRAFFIC study): a randomised trial. Lancet (2002)359:2053-8) and 2.00 minutes in a prior Cilostazol study (Dawson D L, et al. Cilostazol has beneficial effects in treatment of intermittent claudication: results from a multicenter, randomized, prospective, double-blind trial. Circulation (1998)98:678-86). The average of the 2 observed differences is about 1.5 minutes.

Primary efficacy analysis: The primary efficacy variable is tine change in Peak Walking Time (PWT) from baseline to Day 90. Baseline was defined as the average of the 2 qualifying Gardner protocol Exercise Tolerance Tests (ETTs). The treatment effect was evaluated by comparing the difference in the primary efficacy variable between the VLTS-589 treatment group and the placebo treatment group. The primary analysis was based on an analysis of covariance (ANCOVA) to compare the effects of VLTS-589 and placebo on the primary variable. The primary model included main effects due to treatment and center with the baseline value as a covariate. Applicability of the ANCOVA technique was verified before and after unblinding the code. If the model assumptions were not met for the parametric analyses, a proper transformation of the data or a rank ANCOVA, with adjustment for baseline PWT and site, was applied. The parallelism of the 2 treatment regression lines was be assessed. The untransformed ANCOVA analysis was also performed for supporting purposes. The p-values for the comparison of VLTS-589 to placebo, and the 95% confidence interval (CI) for the difference between treatment effects were provided. The p-values of the paired-test and the 95% CI interval for the difference of PWT between baseline and Day 90 within each treatment group were provided. The primary analysis employed observed data. In addition, summary statistics for walking time in minutes was provided.

Analysis of Endpoints: Analysis of the data after decoding showed that the Del-1 formulated drug did not meet its primary endpoint in a Phase II clinical trial in patients with the intermittent claudication form of peripheral arterial disease. The primary efficacy endpoint in the study, improvement in exercise tolerance after 90 days, did not meet statistical significance. However, surprisingly it was appreciated that both the Del-1 and placebo groups showed a statistically significant improvement in exercise tolerance and ankle brachial index (ABI) from baseline. The improvement in both groups was virtually identical.

90 Day Assessment: At the 90-day assessment, the poloxamer (placebo) group of 51 patients had a significant increase in exercise tolerance from baseline of 34% (p<0.00001) and the poloxamer plus Del-1 group of 49 patients had a significant increas e in exercise tolerance from baseline of 32% (p=0.0001). Importantly, the change in ankle brachial index, the clinical indicator of blood flow, was also statistically significant in both groups. In the group receiving poloxamer, there was an increase in ankle brachial index of 0.059 (p=0.00072). For the group receiving poloxamer plus Del-1, there was an increase in ankle brachial index of 0.048 (p=0.00665). Patient demographics and results of secondary endpoints were virtually identical.

The statistically significant effect on exercise tolerance of the poloxamer in the Phase II trial indicated that poloxamer used as a delivery vehicle for the Del-1 gene positively contributed to the exercise tolerance of patients in this trial. Preliminary data for the patients that completed their 180-day assessments indicate their change in exercise tolerance and ABI continued to increase over six months.

180 Day Assessment: As discussed above, at 90 days, there were no significant differences between the treatment groups of poloxamer alone versus poloxamer plus Del-1. However, in both groups, there were significant improvements compared to baseline in exercise tolerance and ankle brachial index (ABI). The primary outcome of the clinical trial was safety and change in PWT (ΔPWT) at 90 days while secondary measures included 180 day ΔPWT, 90 and 180 day ABI, and quality of life measures (QOL).

At 180 day follow up, mean PWT and ABI were increased compared to baseline in both treatment groups with no difference between groups (Table 1) below.

TABLE 1

| | | | | change f/ | P value | P value |
|---|---|---|---|---|---|---|
| | | | 180 | baseline | between | vs. |
| | | Baseline | days | (%) | groups | baseline |
| PWT | VLTS-589 | 5.3 | 7.2 | 34.1 | ns | 0.001 |
| (minutes) | (poloxamer plus Del-1) | | | | | |
| | VLTS-934 | 4.6 | 6.4 | 36.8 | | 0.0002 |
| | (poloxamer only) | | | | | |
| ABI | VLTS-589 | 0.64 | 0.68 | 8.7 | ns | 0.02 |
| | (poloxamer plus Del-1) | | | | | |
| | VLTS-934 | 0.62 | 0.69 | 14.8 | | 0.0003 |
| | (poloxamer only) | | | | | |

In addition, both groups demonstrated significant improvements in QOL measurements vs. baseline, with no significant differences between groups. Serious adverse events were similar in both groups. The conclusion of data analysis is that intramuscular delivery of both the Del-1 with poloxamer and the poloxamer alone resulted in significant improvement in PWT and ABI compared to baseline at 90 and 180 days. There was no difference in outcome measures associated with the Del-1 plasmid supporting a therapeutic effect of the poloxamer rather than a placebo effect in both groups.

Depot delivery: Poloxamer formulations have been utilized for reducing hydrophobic interactions in blood during acute vasocclusive crisis including infarction and sickle cell vaso-occlusive crisis. The role of the poloxamer was to lower blood viscosity, decrease RBC aggregation, and to decrease friction between RBCs and vessel walls, leading to increased microvascular blood flow in ischemic tissues. Uptake into tissues is reported to be minimal and primarily concentrated in highly vascularized tissues. See Gibbs and Hageman, The Annals of Pharmacology 38 (February 2004) 320. In vaso-occlusive crisis, Targe doses are required on the stated basis that small concentrations have little effect on plasma proteins and are not sufficient to systemically activate complement and thus render neutrophils nonresponsive to complement chemotaxis. See U.S. Pat. No. 5,089,260. Furthermore, the polymer is rapidly excreted with a reported half-life of approximately 2 hours such that 90% of an administered dose is excreted in 3 hours. Id For these reasons, the polymer, formulated at a concentration of 150 mg/mL or 15% in buffered saline, is administered by a first large loading dose by bolus IV administration of 100 mg/kg (calculated to be 7 grams in a 70 kg person) followed by continuous infusion of 30/mg/kg/hr for 47 hours (calculated to be 98.7 grams in a 70 kg person) resulting in a total dose of 105.7 grams of poloxamer. Ann. Pharmacother. 38 (2004) 320-4.

In contrast, in one preferred embodiment of the present invention, extravascular depot delivery by multiple intramuscular injections is provided but with a considerably lower acute total body dose than that used in the aforementioned trials.

In one embodiment of the present invention a total IM dose of 2.1 grams is delivered through intramuscular injection of 42 mL of a 5% solution (50 mg/mL) divided into 21 injections in each leg. In another embodiment, extravascular depot delivery by multiple intramuscular injections is provided in which a total IM dose of 4.2 grams is delivered through intramuscular injection of 84 mL of a 5% solution (50 mg/mL) divided into 42 injections, 21 injections per leg in a series of concentric rings in a flow to no flow pattern down each leg.

Therefore the dose of poloxamer 188 in this embodiment is approximately 25 to 50 times lower than the prior intravenous administration in vasocclusive crisis. However, because the poloxamer is delivered by depot administration into an extravascular space in the muscle, the poloxamer is tissue resident for a prolonged period and surprisingly results in improvement in several clinical parameters of peripheral ischemic disease.

Animal Study on Poloxamer in Angiogenesis: Concomitant with the human Phase II clinical trial, an animal study was conducted to assess a variety of morphologic endpoints following intramuscular injection of two dose levels of either saline, poloxamer or poloxamer plus a plasmid encoding Del-1. Normal New Zealand White rabbits were used as the test species. The site of injection was the aggregate musculature of the dorsal lumbar region. Injection was performed, as much as possible, to mimic the application of VLTS-589 in humans. Tissues were collected to permit evaluation of H&E stained sections as well as sections stained to identify endothelial cells (via detection of endogenous alkaline phosphatase and expression of PECAM {CD 31} antigen).

Tissues for H&E staining were collected and fixed in 10% neutral buffered formalin and labeled according to the protocol. Sections were prepared by HCS Laboratories (Evanston, Wash.). The pathologist was unaware of the treatment group assignments during the initial evaluation and grading sequence. Histologic evaluation of muscle sections revealed that although a wide range of vascular density was observed, a consistent pattern was a clear increase in vascular density in the poloxamer only and poloxamer plus plasmid DNA groups with focally abundant endomysial and interstitial capillaries clearly outlining individual muscle fibers at the intramuscular injection sites. This change was easily distinguishable from normal non-injected regions or saline injection sites.

The focal increase in vascular density was not expected in the poloxamer dosed animals and suggests that the polymer provides or facilitates some stimulus that enhances the presence of pericellular vessels. Other than rare, very small mononuclear cell inflammatory cell accumulations there was no histologic evidence of tissue toxicity.

Poloxamer Inhibition of Inflammation in Murine Study: Initial studies incorporating either FGF (positive control), saline (negative control), Del-1 protein (another known angiogenic agent) or poloxamer-188 into Matrigel that was placed subcutaneously into the lower abdomen of mice yielded interesting results. Matrigel Basement Membrane Matrix (BD Biosciences) is a solubilized basement membrane preparation extracted from the Engelbreth-Holm-Swarm (EHS) mouse sarcoma. Its major component is laminin, followed by collagen IV, heparan sulfate proteoglycans, entactin and nidogen. In the Matrigel angiogenesis model, poloxamer-188 was compared to other known angiogenic agents such as fibroblast growth factor (FGF, positive control), Del-1 protein, or saline as a negative control.

This angiogenesis model demonstrated prolific new vascular growth for FGF, light to moderate vessel growth for Del-1, and a slight lamellar pattern for P188 and saline. After gaining experience with the system, it was felt that the quantity of Del-1 used should be titrated to see if a dose response with the protein could be distinguished, and to alter the ratio of P188 to matrigel. In the first round of studies it was noted that the 5% P188 concentration added to the Matrigel appeared to inhibit polymerization of the Matrigel. An in vitro titrational study showed that when matrigel was mixed with either 1% or 2% P188 polymerization of the Matrigel was normal. At 3% P188 concentration the Matrigel underwent clumping polymerization, and 5% it was almost completed inhibited. Therefore, in the repeat in vivo experiment the Matrigel and poloxamer concentrations were altered such that the initial concentrations were similar to those that yielded good polymerization, but still gave a final concentration of 5% P188 (the concentration tested in clinical trials).

Matrigel implants were placed subcutaneously in the lower abdominal/inguinal region of mice and harvested 4 to 7 days later. Following fixation in 10% neutral buffered formalin implants were embedded in paraffin and stained with H&E. Histological examination of implants formulated with various concentrations of Del-1 protein, Del-1 protein with Poloxamer 188, FGF protein, Poloxamer 188 (various ratios) or Poloxamer with saline revealed three distinct morphologic patterns.

The first of these was a pattern characterized by high cellularity with the Matrigel matrix being displaced and infiltrated by mesenchymal cells, small blood vessels and variable numbers of inflammatory cells. The infiltrating cells resulted in the presence of isolated Matrigel islands or trabeculae or, on occasion, scattered, isolated foci of mesenchymal cells. This pattern was typical of Matrigel containing either FGF or Del-1 protein at a concentration of 200 ug/ml. The cellular response of the Del-1 differed slightly from the FGF in that the Del-1 response was very slightly less intense and contained more neutrophils than the FGF implants. Concentrations of Del-1 less than 200 ugs/ml displayed substantially less cellular response.

The second response was a pattern characterized by markedly reduced cellularity with preservation of broad sheets of Matrigel matrix and little peripheral response. What cellular response was present was characterized by a variably thick fibrous capsule that surrounded portions of the matrix. Rare individual clusters of mesenchymal cells were occasionally contained within the matrix. Likewise, inflammatory cells were rare within or surrounding the matrix. This pattern was present whenever Poloxamer 188 was present either as a solitary component or when formulated with Del-1 (addition of Del-1 slightly enhanced the cellularity but the change was minor). This pattern was consistent with Poloxamer 188 displaying a substantial anti-inflammatory effect including a reduction in inflammatory, neovascular and fibrous tissue responses.

The third pattern of response was characterized by the formation of a laminar pattern. In this pattern the laminations in the Matrigel were formed by the infiltration of spindle cells (resembling fibroblasts) between sheets of matrigel matrix. This was frequently accompanied by the presence of a variably thick fibrous capsule surrounding the primary implant. Small clusters of mixed inflammatory cells were present in a few peripheral sites but this was not a common occurrence. This laminar pattern was exclusive to Matrigel formulated with saline.

In conclusion, poloxamer 188 inhibited the inflammatory reaction induced both by foreign proteins as well as capsule formation surrounding the implantation of a foreign body having low inherent antigenicity.

The foregoing disclosure and description of the invention are illustrative and explanatory thereof, and various changes in the size, shape, and materials, as well as in the details of the illustrated system may be made without departing from the spirit of the invention. The invention is claimed using terminology that depends upon a historic presumptive presentation that recitation of a single element covers one or more, and recitation of two elements covers two or more, and the like.

What is claimed is:

1. A method for treatment of a symptom of tissue inflammation comprising local depot administration to an affected tissue of a composition comprising an effective amount of a poloxamer.

2. The method of claim 1, wherein the poloxamer is administered at a concentration of about 0.1 to 100%.

3. The method of claim 2, wherein the poloxamer has a hydrophilic component of about 80% or greater and a hydrophobic molecular weight between 950 and 4000 daltons.

4. The method of claim 3, wherein the poloxamer has the copolymer structure, physical form and surfactant characteristic of poloxamer-188.

5. The method of claim 3, wherein the poloxamer is administered at concentration of between about 0.1 and 20% w/v.

6. The method of claim 5, wherein the poloxamer 188 is administered at a concentration of about 1-15%.

7. The method of claim 6, wherein the composition consists essentially of 50 mg/ml w/v poloxamer-188, 0.28 mg/ml w/v of Tris, and 0.44 mg/ml of Tris-HCl in an aqueous saline solution.

8. The method of claim 1, wherein the composition is locally administered for depot in an extravascular tissue by intramuscular, intravascular and/or intracapsular injection.

9. The method of claim 8, wherein the intramuscular injection involves a plurality of injections.

10. The method of claim 1, wherein the tissue inflammation is associated with tissue ischemia in peripheral vascular, cardiovascular, cerebrovascular and renovascular disease.

11. The method of claim 1, wherein the composition further comprises one or more biological agents that are able to stimulate the growth and maturation of new collateral vessels in the affected tissue.

12. The method of claim 1, wherein the composition is lyophilized for storage and is rehydrated prior to administration.

13. A method of reducing local production of at least one inflammatory cytokine comprising local administration of an effective amount of a poloxamer into a tissue affected by an inflammatory process.

14. The method of claim 13, wherein the poloxamer has a copolymer structure, physical form and surfactant characteristic of a poloxamer-188.

15. A method of reducing local production of at least one inflammatory mediator comprising local administration into a tissue of an effective amount of a poloxamer, wherein the poloxamer has a hydrophilic component of about 80% or greater and a hydrophobic molecular weight between 950 and 4000 daltons.

16. The method of claim 15, wherein the poloxamer is present in an aqueous solution at a concentration of between about 0.1 and about 20% w/v.

17. The method of claim 16, wherein the poloxamer has a copolymer structure, physical form and surfactant characteristic of a poloxamer-188.

18. The method of claim 15, wherein the inflammatory mediator is at least one of: IL-6, IL-8, MCP-1, and GRO.

19. The method of claim 16, wherein the aqueous solution further comprises one or more pharmacologic excipients.

20. The method of claim 15, wherein the local administration is for deposition in an extravascular tissue by intramuscular, intravascular and/or intracapsular injection.
